(19) United States
(12) Patent Application Publication
(10) Pub. No.: US 2024/0409968 A1
(43) Pub. Date: Dec. 12, 2024

(54) Enriched Microbial Biocatalyst for Conversion of CO2 into Acetate

(71) Applicant: United States Department of Energy, Washington, DC (US)

(72) Inventors: Djuna Gulliver, Pittsburgh, PA (US); Daniel Ross, Pittsburgh, PA (US); Samuel Flett, Pittsburgh, PA (US)

(21) Appl. No.: 18/732,013

(22) Filed: Jun. 3, 2024

Related U.S. Application Data
(60) Provisional application No. 63/471,809, filed on Jun. 8, 2023.

(51) Int. Cl. C12P 7/54

(52) U.S. Cl. CPC C12P 7/54

(57) ABSTRACT

One or more embodiments relate to a method for converting a carbon feedstock into more desirable products involving contacting a biocatalyst with a carbon feedstock, where the biocatalyst converts the carbon feedstock into more desirable products, and where the biocatalyst features Peptostreptococcales-Tissierellales, Proteiniclasticum, Proteiniphilum, Mesobacillus, Acetobacterium, and combinations thereof.

### CROSS-REFERENCE TO RELATED APPLICATIONS

This Utility Patent application claims priority benefit as a U.S. Non-Provisional of U.S. Provisional Patent Application Ser. No. 63/471,809, filed on Jun. 8, 2023, currently pending, the entirety of which is incorporated by reference herein.

### STATEMENT OF GOVERNMENT SUPPORT

The United States Government has rights in this invention pursuant to the employer-employee relationship of the Government to the inventors as U.S. Department of Energy employees and site-support contractors at the National Energy Technology Laboratory.

### FIELD OF THE INVENTION

Embodiments relate to bioconversion of carbon feedstocks into more desirable compounds. Specifically, embodiments relate to using a mixed microbial community as a biocatalyst to convert C1 feedstocks into more desirable products.

### BACKGROUND

With the continual increase in anthropogenic CO₂ emissions, and subsequent global warming, it is necessary to strive towards a low-carbon economy, particularly non-fossil carbon sources for chemical and fuel production. To reduce greenhouse gas emissions, CO₂ utilization technologies have been developed to capture, store, and/or convert waste carbon, including electrochemical conversion, thermo/chemical conversion (Fisher-Tropsch), and biological conversion. While direct reduction of CO₂ via non-biological routes requires high energy inputs (e.g., elevated temperature and pressure), have a limited product spectrum, and low product selectivity, the latter biological approach exploits the natural ability of microorganisms to capture and utilize gaseous one-carbon (C1) compounds at atmospheric pressure and near-ambient temperatures.

Microorganisms can utilize various gaseous carbon substrates including industrial waste gasses, synthesis gas (H and CO), and carbon monoxide. Liquid C1 carbon compounds also serve as substrates for microbial conversion, including formaldehyde, methyl groups, methanol, and formate. Bioconversion of gaseous and liquid C1 carbon substrates produces various acids, alcohols, and diols, including acetate, butyrate, caproate, lactate, ethanol, n-butanol, n-hexanol, and 2,3-butanediol using native pathways. Utilization of engineered (non-native) pathways increases the product spectrum to include other acids (succinate, mevalonate, 3-hydroxybutyrate, 3-hydroxypropionate, polyhydroxybutyrate), alcohols (isopropanol, 2-butanol), diols (1,3-butanediol), ketones (acetone, methyl ethyl ketone), aromatics (2-phenylethanol, para-hydroxybenzoate), and terpenoids (isoprene, farnesene).

While individual strains (Acetobacterium woodii, Clostridium autoethanogenum, Clostridium ljungdahlii, Clostridium carboxydivorans, Clostridium ragsdalei, Eubacterium limosum, and Moorella thermoacetica, among others) are capable of high production rates, and amendable to genetic modification to enhance titers and produce non-native products, mixed microbial communities are often more resistant to contamination, contain functional redundancy, and are more resilient against perturbations in operational parameters. Therefore, there is a need in the art for a mixed microbial community for the production of short-chain organic acids from H₂:CO₂.

### SUMMARY

One object of at least one embodiment of the present invention is related to providing a method for converting C1 compounds into more desirable products using a biocatalyst.

Another object of at least one embodiment of the present invention is related to a mixed microbial community comprising a biocatalyst suitable for converting CO₂ and other C1 compounds into more desirable products/compounds.

Still yet another object of at least one embodiment of the present invention is related to providing a mixed microbial community comprising a biocatalyst suitable to convert different C1 compounds into more desirable products/compounds, the biocatalyst capable of adapting to conversion of different feedstocks without adjustment.

The invention provides a method for converting a carbon feedstock into more desirable products comprising contacting a biocatalyst with a carbon feedstock, wherein the biocatalyst converts the carbon feedstock into more desirable products, and wherein the biocatalyst comprises bacteria selected from the group consisting of Peptostreptococcales-Tissierellales, Proteiniclasticum, Proteiniphilum, Mesobacillus, Acetobacterium, and combinations thereof.

### BRIEF DESCRIPTION OF THE DRAWINGS

The invention together with the above and other objects and advantages will be best understood from the following detailed description of the preferred embodiment of the invention shown in the accompanying drawings, wherein:

FIG. 1 is a flowchart for a method of converting a carbon feedstock into more desirable products using a mixed microbial community as a biocatalyst, in accordance with the features of the present invention;

FIG. 2 is a flowchart for generating the invented biocatalyst, in accordance with the features of the present invention;

FIG. 3A is a plot of the taxonomy of the microbial community in two coal seam produced water samples, in accordance with the features of the present invention;

FIG. 3B is growth profiles of the microbiome of coal seam produced water; in accordance with the features of the present invention;

FIG. 3C is growth profiles of enrichment cultures incubated for seven days at room temperature, 30° C., and 40° C., all with no agitation, in accordance with the features of the present invention;

FIG. 3D is growth profiles of enrichment cultures incubated for seven days at room temperature, 30° C., and 40° C., all with 200 rpm agitation, in accordance with the features of the present invention;

FIG. 3E is acetate concentrations at day 7 for incubations of enrichment cultures at room temperature, 30° C., and 40° C., with addition of 0.5, 1, or 2 g/L yeast extract, in accordance with the features of the present invention;

FIG. 3F is a plot of relative abundance of microbial taxa in the 7 day incubations shown in FIG. 3E; in accordance with the features of the present invention;

FIG. 4A is a plot of optical density at 600 nm for two operating bioreactors using the invented biocatalyst, in accordance with the features of the present invention;

FIG. 4B is a plot of pH for two operating bioreactors using the invented biocatalyst, in accordance with the features of the present invention;

FIG. 4C is a plot of acetate concentration in two operating bioreactors using the invented biocatalyst, in accordance with the features of the present invention;

FIG. 4D is a plot of relative abundance of microbial taxa in two operating bioreactors using the invented biocatalyst, in accordance with the features of the present invention;

FIG. 4E is a plot of relative abundance of microbial taxa in two operating bioreactors using the invented biocatalyst based on shotgun metagenome sequencing, in accordance with the features of the present invention;

FIG. 4F is a plot of a quality assessment of metagenome-assembled genomes for the invented biocatalyst used in operating bioreactors, in accordance with the features of the present invention;

FIG. 5A is a growth profile of the invented catalyst while grown for 7 days on N₂ and 0, 50, 100, 200, and 400 mM formate in a bioreactor, in accordance with the features of the present invention;

FIG. 5B is a plot of the pH inside the bioreactor after growing the invented biocatalyst for 7 days on N₂ and 0, 50, 100, 200, and 400 mM formate in a bioreactor, in accordance with the features of the present invention;

FIG. 5C is a plot of acetate concentration inside the bioreactor after growing the invented biocatalyst for 7 days on N₂ and 0, 50, 100, 200, and 400 mM formate in a bioreactor, in accordance with the features of the present invention;

FIG. 5D is a plot of formate concentration inside the bioreactor after growing the invented biocatalyst for 7 days on N₂ and 0, 50, 100, 200, and 400 mM formate in a bioreactor, in accordance with the features of the present invention;

FIG. 5E is a plot showing amplicon-based taxonomy of the invented biocatalyst after growing for 7 days on N₂:CO₂ (80:20) and 0, 50, 100, 200, and 400 mM formate in a bioreactor, in accordance with the features of the present invention;

FIG. 5F is a growth profile of the invented catalyst while grown for 7 days on N₂:CO₂ (80:20) and 0, 50, 100, 200, and 400 mM formate in a bioreactor, in accordance with the features of the present invention;

FIG. 5G is a plot of the pH inside the bioreactor after growing the invented biocatalyst for 7 days on N₂:CO₂ (80:20) and 0, 50, 100, 200, and 400 mM formate in a bioreactor, in accordance with the features of the present invention;

FIG. 5H is a plot of acetate concentration inside the bioreactor after growing the invented biocatalyst for 7 days on N₂:CO₂ (80:20) and 0, 50, 100, 200, and 400 mM formate in a bioreactor, in accordance with the features of the present invention;

FIG. 5I is a plot of formate concentration inside the bioreactor after growing the invented biocatalyst for 7 days on N₂:CO₂ (80:20) and 0, 50, 100, 200, and 400 mM formate in a bioreactor, in accordance with the features of the present invention;

FIG. 5J is a plot showing amplicon-based taxonomy of the invented biocatalyst after growing for 7 days on N₂:CO₂ (80:20) and 0, 50, 100, 200, and 400 mM formate in a bioreactor, in accordance with the features of the present invention;

FIG. 5K is a growth profile of the invented catalyst while grown for 7 days on H₂:CO₂ (80:20) and 0, 50, 100, 200, and 400 mM formate in a bioreactor, in accordance with the features of the present invention;

FIG. 5L is a plot of the pH inside the bioreactor after growing the invented biocatalyst for 7 days on H₂:CO₂ (80:20) and 0, 50, 100, 200, and 400 mM formate in a bioreactor, in accordance with the features of the present invention;

FIG. 5M is a plot of acetate concentration inside the bioreactor after growing the invented biocatalyst for 7 days on H₂:CO₂ (80:20) and 0, 50, 100, 200, and 400 mM formate in a bioreactor, in accordance with the features of the present invention;

FIG. 5N is a plot of formate concentration inside the bioreactor after growing the invented biocatalyst for 7 days on H₂:CO₂ (80:20) and 0, 50, 100, 200, and 400 mM formate in a bioreactor, in accordance with the features of the present invention;

FIG. 5O is a plot showing amplicon-based taxonomy of the invented biocatalyst after growing for 7 days on H₂:CO₂ (80:20) and 0, 50, 100, 200, and 400 mM formate in a bioreactor, in accordance with the features of the present invention;

FIG. 5P shows an amplicon-based taxonomy of the invented biocatalyst grown with formate and N₂, N₂:CO₂, or H₂:CO₂, in accordance with the features of the present invention;

FIG. 5Q shows a quality assessment of metagenome-assembled genomes for the biocatalyst grown with formate and N₂, N₂:CO₂, or H₂:CO₂, in accordance with the features of the present invention;

FIG. 6A is a growth profile of the invented catalyst grown over various gasses at 30° C. for 7 days on various gasses at 30° C. with 200 rpm agitation, in accordance with the features of the present invention;

FIG. 6B is a plot of the pH inside the bioreactor after growing the invented biocatalyst for 7 days on various gasses at 30° C. with 200 rpm agitation, in accordance with the features of the present invention;

FIG. 6C is a plot of acetate concentration inside the bioreactor after growing the invented biocatalyst for 7 days on various gasses at 30° C. with 200 rpm agitation, in accordance with the features of the present invention;

FIG. 6D is a plot showing amplicon-based taxonomy of the invented biocatalyst after growing for 7 days on various gasses at 30° C. with 200 rpm agitation, in accordance with the features of the present invention;

FIG. 6E is a growth profile of the invented catalyst grown over various gasses at 30° C. while grown for 7 days on various gasses and 200 mM formate at 30° C. with 200 rpm agitation, in accordance with the features of the present invention;

FIG. 6F is a plot of the pH inside the bioreactor after growing the invented biocatalyst for 7 days on various gasses and 200 mM formate at 30° C. with 200 rpm agitation, in accordance with the features of the present invention;

FIG. 6G is a plot of acetate concentration inside the bioreactor after growing the invented biocatalyst for 7 days on various gasses and 200 mM formate at 30° C. with 200 rpm agitation, in accordance with the features of the present invention;

FIG. 6H is a plot of formate concentration inside the bioreactor after growing the invented biocatalyst for 7 days on various gasses and 200 mM formate at 30° C. with 200 rpm agitation, in accordance with the features of the present invention;

FIG. 6I is a plot showing amplicon-based taxonomy of the invented biocatalyst after growing for 7 days on various gasses and 200 mM formate at 30° C. with 200 rpm agitation, in accordance with the features of the present invention;

FIG. 7A is a growth profile of the invented catalyst grown over a N₂:CO₂ mixture (80:20) with 0, 1.25, 2.5, 5, or 10 g/L NaHCO₃ for 7 days, in accordance with the features of the present invention;

FIG. 7B is a growth profile of the invented catalyst grown over a N₂:CO₂ mixture (80:20) with 200 mM formate and with 0, 1.25, 2.5, 5, or 10 g/L NaHCO₃ for 7 days, in accordance with the features of the present invention;

FIG. 7C is a growth profile of the invented catalyst grown over a H₂:CO₂ mixture (80:20) with 0, 1.25, 2.5, 5, or 10 g/L NaHCO₃ for 7 days, in accordance with the features of the present invention;

FIG. 7D is a growth profile of the invented catalyst grown over a H₂:CO₂ mixture (80:20) with 200 mM formate and with 0, 1.25, 2.5, 5, or 10 g/L NaHCO₃ for 7 days, in accordance with the features of the present invention;

FIG. 7E is a plot of the pH inside the bioreactor after growing the invented biocatalyst over a N₂:CO₂ mixture (80:20) with 0, 1.25, 2.5, 5, or 10 g/L NaHCO₃ for 7 days, in accordance with the features of the present invention;

FIG. 7F is a plot of the pH inside the bioreactor after growing the invented biocatalyst over a N₂:CO₂ (80:20) mixture with 200 mM formate and with 0, 1.25, 2.5, 5, or 10 g/L NaHCO₃ for 7 days, in accordance with the features of the present invention;

FIG. 7G is a plot of the pH inside the bioreactor after growing the invented biocatalyst over a H₂:CO₂ (80:20) mixture with 0, 1.25, 2.5, 5, or 10 g/L NaHCO₃ for 7 days, in accordance with the features of the present invention;

FIG. 7H is a plot of the pH inside the bioreactor after growing the invented biocatalyst over a H₂:CO₂ mixture (80:20) with 200 mM formate and with 0, 1.25, 2.5, 5, or 10 g/L NaHCO₃ for 7 days, in accordance with the features of the present invention;

FIG. 7I is a plot of the acetate concentration inside the bioreactor after growing the invented biocatalyst over a N₂:CO₂ mixture (80:20) with 0, 1.25, 2.5, 5, or 10 g/L NaHCO₃ for 7 days, in accordance with the features of the present invention;

FIG. 7J is a plot of the acetate concentration inside the bioreactor after growing the invented biocatalyst over a N₂:CO₂ (80:20) mixture with 200 mM formate and with 0, 1.25, 2.5, 5, or 10 g/L NaHCO₃ for 7 days, in accordance with the features of the present invention;

FIG. 7K is a plot of the acetate concentration inside the bioreactor after growing the invented biocatalyst over a H₂:CO₂ mixture (80:20) with 0, 1.25, 2.5, 5, or 10 g/L NaHCO₃ for 7 days, in accordance with the features of the present invention;

FIG. 7L is a plot of the acetate concentration inside the bioreactor after growing the invented biocatalyst over a H₂:CO₂ mixture (80:20) with 200 mM formate and with 0, 1.25, 2.5, 5, or 10 g/L NaHCO₃ for 7 days, in accordance with the features of the present invention;

FIG. 7M is a plot showing amplicon-based taxonomy of the invented biocatalyst after growing the invented biocatalyst over a N₂:CO₂ mixture (80:20) with 0, 1.25, 2.5, 5, or 10 g/L NaHCO₃ for 7 days with and without formate, in accordance with the features of the present invention; and

FIG. 7N is a plot showing amplicon-based taxonomy of the invented biocatalyst after growing the invented biocatalyst over a H₂:CO₂ mixture (80:20) with 0, 1.25, 2.5, 5, or 10 g/L NaHCO₃ for 7 days with and without formate, in accordance with the features of the present invention;

The invention together with the above and other objects and advantages will be best understood from the following detailed description of the preferred embodiment of the invention shown in the accompanying drawings, wherein:

### DETAILED DESCRIPTION

The foregoing summary, as well as the following detailed description of certain embodiments of the present invention, will be better understood when read in conjunction with the appended drawings.

As used herein, a C1 compound comprises a carbon containing compound having a single carbon and or a single carbon-length chain and or a methyl group on a larger compound.

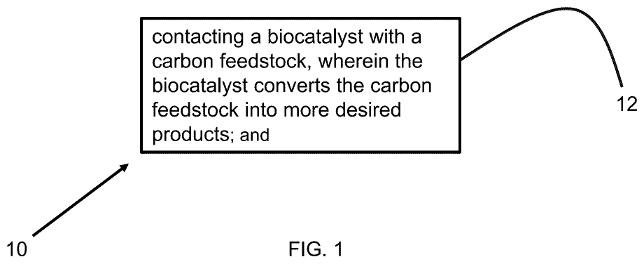

In an embodiment, the invention provides a method for converting a carbon feedstock into more desirable products 10, the method 10 shown in the flowchart of FIG. 1. As shown in FIG. 1, the method 10 begins by contacting a biocatalyst with a carbon feedstock 12, wherein the biocatalyst converts the carbon feedstock into the more desirable products. The contacting step 12 is performed at a contacting temperature.

In the method 10, the carbon feedstock is any carbon-containing compound suitable for conversion by the biocatalyst into more desirable products. In an embodiment, the feedstock is a compound or mixture of compounds comprising one or more C1 compounds suitable for conversion into more desirable products. In an embodiment, the C1 compounds in the feedstock are gaseous, liquid, and combinations thereof. Exemplary and suitable C1 compounds comprise CO₂, formate, methanol, CO, methyl groups on larger compounds, and combinations thereof. These C1 compounds are exemplary and not meant to be limiting. A person having ordinary skill in the art will readily ascertain that the invented method is suitable for converting any C1 compounds that can be converted by the biocatalyst into more desirable products.

In some embodiments, the contacting step 12 comprises contacting the biocatalyst with a feedstock comprising at least one gaseous C1 compound selected from CO₂, CO, methyl groups on larger gas-phase compounds, and combinations thereof. In such an embodiment, the contacting step 12 comprises delivering the one or more C1 compounds as a gaseous stream to a bioreactor containing the invented biocatalyst. In embodiments, the gaseous stream also comprises other gasses selected from N₂, H₂, various other gasses, and combinations thereof.

In another embodiment, the feedstock comprises a liquid C1 compound. Suitable liquid C1 compounds comprise compounds selected from formate, methanol, and combinations thereof. Additional liquid C1 compounds include methyl groups of larger liquid-phase compounds. In embodiments, where using a liquid C1 compound in the feedstock, the contacting step 12 comprises dispersing the liquid C1 compound in a bioreactor containing the biocatalyst, wherein the liquid C1 compound serves as a substrate and or growth medium for the biocatalyst. In an embodiment, a liquid C1 compound or compounds are added to the bioreactor in any concentration suitable for use in the instant method, wherein concentration of liquid C1 compound in the bioreactor comprises the concentration of said liquid C1 compound or compounds calculated according to the liquid contents of said bioereactor. In an exemplary embodiment, the invented biocatalyst is suitable to use formate having concentrations ranging between approximately 50 mM to approximately 1 M, preferably between approximately 50 mM to approximately 400 mM in the bioreactor.

In yet another embodiment, the feedstock comprises both liquid and gaseous C1 compounds. In such embodiments, the contacting step 12 comprises a combination of C1 compound delivery methods described in the preceding paragraphs.

As described above and shown in FIG. 1, the invented method produces more desirable products from the C1 compounds in the feedstock. In an embodiment, the more desirable products are any conversion products made from the biocatalyst after contact with the C1 compounds. In another embodiment, the more desirable products are short chain organic compounds. Specific and exemplary more desirable products comprise acetate, butyrate, isovaleric acid, hexanoic acid, phenyl acetic acid, various other acids, alcohols, and diols, including caproate, lactate, ethanol, n-butanol, n-hexanol, and 2,3-butanediol, other acids (succinate, mevalonate, 3-hydroxybutyrate, 3-hydroxypropionate, polyhydroxybutyrate), alcohols (isopropanol, 2-butanol), diols (1,3-butanediol), ketones (acetone, methyl ethyl ketone), aromatics (2-phenylethanol, para-hydroxybenzoate), terpenoids (isoprene, farnesene) and combinations thereof. These products are exemplary and not meant to be limiting. A person having ordinary skill in the art could readily ascertain that the invented method is suitable for converting C1 into a multitude or products depending on the feedstock. These products are advantageous over prior art processes that primarily make ethanol. In an embodiment, the more desired products listed in this paragraph are ions such as acetate and formate. In such embodiments, the more desired products also include those compounds in their fully protonated form, i.e. acetic acid and formic acid.

A salient feature of the invention is the biocatalyst used to convert the carbon feedstock into more desirable products. In an embodiment, the biocatalyst is a mixed microbial community of wild-type bacteria that has been modified to convert C1 compounds into more desirable products and to be adaptable to converting different C1 compounds into more desirable products. In an embodiment, the biocatalyst comprises bacteria selected from the group consisting of Peptostreptococcales-Tissierellales, Proteiniclasticum, Proteiniphilum, Mesobacillus, Acetobacterium, and combinations thereof. In an embodiment, the biocatalyst comprises a combination of Peptostreptococcales-Tissierellales, Proteiniclasticum, Proteiniphilum, Mesobacillus, Acetobacterium in varying concentrations. In an embodiment, the relative concentration of the various bacteria comprising the biocatalyst adapts to the feedstock being converted to more desirable products, i.e. the concentration of bacteria best able to convert a particular feedstock increases relative to other bacteria comprising the biocatalyst. In an embodiment, the mixed microbial community comprising the biocatalyst is configured to adapt to different feedstocks, wherein adapting to different feedstocks comprises an increase in concentration of bacteria best able to convert a particular feedstock relative to other bacteria comprising the biocatalyst.

Significantly, the bacteria comprising the biocatalyst are wild-type strains that have not been genetically modified. This feature provides several advantages over prior art biocatalysts using genetically modified organisms. Namely, the instant biocatalyst is less sensitive to feedstock content than prior art methods using genetically modified organisms. Further, the instant biocatalyst, as a result of utilizing wild-type bacteria, is less sensitive to stressors, requires less condition control, does not suffer from strain stability, and has reduced sensitivity to contamination over prior art methods using genetically modified bacteria strains. A salient feature of the invention is the improvement and modification of the originally sampled bacteria community through continuous transfer to generate the invented biocatalyst. Said improvement and modification provides a biocatalyst that is adaptable to convert different C1 feedstocks as described above, and, overall, provides a biocatalyst that is superior in conversion of C1 feedstocks to more desirable compounds. Said improvement and modification provides a biocatalyst superior to and not available in nature. In an embodiment, the finalized biocatalyst has a reduced number of members (types of bacterial strains) than the original bacterial community found in the wild, wherein the finalized biocatalyst is also superior in its ability to convert C1 feedstocks, adapt to differing feedstocks, and, in an embodiment, some of the bacteria comprising the biocatalyst have genetic modifications as a result of the steps taken to generate the biocatalyst from the originally collected bacteria community.

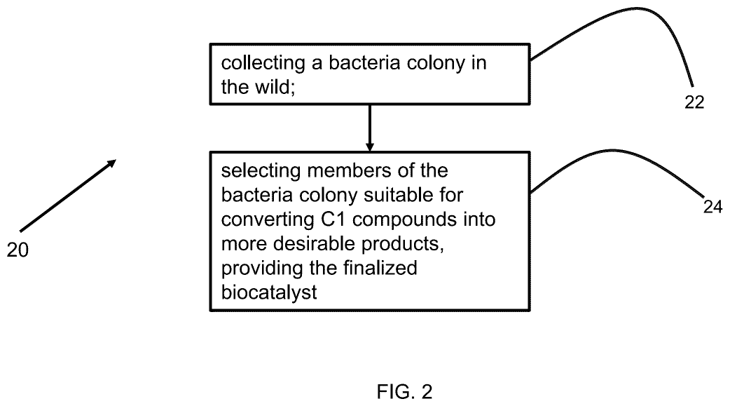

In an embodiment, the biocatalyst comprises a microbial community developed from a sample bacteria community found in the wild. In such an embodiment, the method 10 shown in FIG. 1 is preceded by a generating a biocatalyst step. This generating step 20 is shown in the flowchart shown in FIG. 2. The generating a biocatalyst step begins with collecting a bacteria community in the wild 22. The generating step 20 continues with selecting members of the bacteria community suitable for converting C1 compounds into more desirable products 24, providing the finalized biocatalyst. In an embodiment, the selecting members step 24 comprises manipulating the bacteria community collected from the wild to increase the concentration of bacteria suitable for converting C1 compounds into more desirable products and reducing and or removing the concentration of bacteria less and or not suitable for converting C1 compounds into more desirable products. Specifically, in an embodiment, the selecting members step 24 comprises continual transfer on anaerobic media.

The invented biocatalyst has the distinct advantage over the prior art in that it is flexible in that it is suitable for and configured to convert different C1 compounds into products without adjustment of said biocatalyst. In an embodiment, the biocatalyst is configured to convert more than one C1 compound into more than one product, wherein the conversion of different C1 compounds into products occurs while the different C1 compounds are supplied simultaneously or in batches, and without adjusting the biocatalyst. In an embodiment, the biocatalyst is adaptable to convert any C1 compound into more desirable products without adjustment.

Returning to FIG. 1, in the invented method 10 shown in FIG. 1, the biocatalyst is situated in a bioreactor. In an embodiment, the bioreactor comprises any bioreactor suitable to contact the biocatalyst with C1 compounds. Where the C1 compounds of the feedstock are gaseous, the contacting step 12 comprises proving said gaseous C1 compounds to the bioreactor via a gas inlet continuously and or batchwise. In an embodiment where the C1 compounds contacting the biocatalyst are liquid, the contacting step 12 comprises providing said liquid C1 compounds to an interior of the bioreactor such that they are in contact with the biocatalyst. In an embodiment, where a combination of gaseous and liquid C1 compounds are used in the contacting step 12, the gaseous C1 compounds are provided to the bioreactor via a gas inlet and the liquid C1 compounds are provided to the interior of the bioreactor such that the liquid C1 compounds are in contact with the biocatalyst. In an embodiment where both gaseous and liquid C1 compounds are provided in the contacting step 12, both, both phases of C1 compounds are added batch wise and or continuously, wherein, in an embodiment, the phases of C1 compounds are added to the bioreactor independently such that liquid phase C1 compounds are added to the bioreactor continuously or batch wise independently of whether the gas phase C1 compounds are added continuously or batch wise. Embodiments of the invention utilize growth media in the bioreactor, wherein said growth media comprises materials selected from sodium bicarbonate, yeast extract, ATCC trace mineral mix, ATCC vitamin mix, ammonium chloride, potassium phosphate, and magnesium chloride, and combinations thereof. A person having ordinary skill in the art will recognize that this list is exemplary and not meant to be limiting. Any growth suitable for use with the invented biocatalyst in converting C1 compounds to more desirable compounds may be used with the invented method.

Another salient feature of the invention is the contacting temperature. State of the art bio methods to convert C1 compounds require elevated contacting temperatures. In an embodiment, the instant method features a contacting step 12 carried out at lower contacting temperatures and pressures compared to the prior art, requiring only standard temperature and pressure. In an embodiment, the contacting step 12 is carried out at any contacting temperature suitable for the biocatalyst to convert C1 compounds in the feedstock into more desirable compounds. Exemplary and suitable contacting temperatures are between approximately 25° C. and approximately 40° C., preferably between approximately 25° C. and approximately 37° C. In an embodiment, the contacting temperature is room temperature. Prior art methods often require much higher operating temperatures between 5° and 70° C.

Yet another salient feature of the invention is the high conversion percentage of the C1 compounds in the feedstock into more desirable compounds. In an embodiment, the invented method converts between approximately 50% and approximately 100% of C1 compounds into more desirable products without adding more biocatalyst, preferably between approximately 80% and approximately 100%, and typically over 80%. In an embodiment, the invented method converts 100% of C1 compounds into more desirable products.

Still yet another salient feature of the invention is the yields of desirable products. In an embodiment, the invented method generates between approximately 1 and approximately 7 g/L of acetate within the bioreactor.

Another salient feature of the invention is that the invented method is not an electrochemical method. Prior art use microbial communities only in electrochemical methods. In an embodiment, the instant invention does not use the invented catalyst in an environment with a voltage bias and therefore does not take place in an electrochemical cell.

### Examples

### Wild-Type Bacteria Community

The inoculum (bacteria community collected from the wild) for enrichment cultures originated from samples obtained from a coal seam methane well located in Southwestern Pennsylvania, USA. Water pumped from the well was immediately collected in sterile 1 L bottles and analyzed for pH, temperature, conductivity, and TDS using a multi-parameter water quality checker (Horiba Instruments, Japan). Produced water samples were placed on ice for transport. Samples were collected in June and December of 2015. Water temperature for June and December was 23.9° C. to 8.24° C., respectively. The pH ranged from 7.16 to 7.54, conductivity (mS/cm) ranged from 9.43 to 55.3, and TDS (g/L) ranged from 5.94 to 33.1.

### Medium Composition

‘FW’ medium: A freshwater medium ‘FW’ was prepared according to Marshall C W et al., 2013, Environmental Science & Technology 47:6023-6029, the entirety of which is incorporated by reference herein, and contained the following (per liter): 0.25 g NH₄Cl, 0.1 g KCl, 0.212 g MgCl₂, 0.03 g CaCl₂), 0.6 g NaH₂PO₄, 2.5 g NaHCO₃, 20 mL ATCC Vitamin Supplement, and 20 mL ATCC Trace Mineral Supplement.

Phosphate medium: A phosphate-buffered medium was prepared according to LaBelle E V et al., 2014, PLOS ONE 9:e109935, the entirety of which is incorporated by reference herein, and contained the following (per liter): 0.25 g NH₄Cl, 0.1 g KCl, 0.212 g MgCl₂, 0.03 g CaCl₂), 0.6 g NaH₂PO₄, 5.24 g KH₂PO₄, 10.71 g K₂HPO₄, 2.92 g NaCl, 20 mL ATCC Vitamin Supplement, and 20 mL ATCC Trace Mineral Supplement.

DSMZ 135 medium: A modified version of DSMZ 135 medium was prepared according to Kracke F. et al., 2019, Communications Chemistry 2:45, the entirety of which is incorporated by reference herein. DSMZ 135 medium contained the following (per liter): 1 g NH₄Cl, 0.33 g KH₂PO₄, 0.45 g K₂HPO₄, 0.1 g MgSO₄, 2 g yeast extract, 10 g NaHCO₃, 0.5 g L-cysteine, 0.5 g Na₂S, 20 mL ATCC Vitamin Supplement, and 20 mL ATCC Trace Mineral Supplement. The following modifications were made: 100 mM MOPS buffer (pH7) was added, Na-resazurin, Na₂S, and fructose were omitted, and the concentration of L-cysteine was increased from 0.5 g/L to 1 g/L.

### Growth and Extracellular Metabolite Analysis of Cultures.

Cell density was measured using the optical density at 600 nm absorbance with a Spectronic 200 spectrophotometer (Thermo Scientific). Fatty acids and alcohols from liquid supernatant were identified and quantified using high pressure liquid chromatography (HPLC). On day 7, supernatant was extracted from anaerobic tubes, filtered (0.25 μm), and loaded into the HPLC autosampler. The HPLC (Shimadzu) was operated at 0.55 mL/min, using an Aminex HPX-87H column (Bio-Rad, Hercules, CA, USA) with a column temperature of 40° C. Organic acids were monitored using a refractive index detector (RID) and a photodiode array detector (PDA) at 210 nm. The mobile phase was 5 mM sulfuric acid. Standards were diluted in deionized water or modified DSMZ 135 medium. All samples (performed in triplicate reactors) and standards were analyzed in duplicate (technical replicates).

DNA extraction, 16S rRNA gene sequencing, and amplicon processing

On day 7 a sample of cell culture (1.2 mL) was extracted and spun at 13,500 rpm for 5 min. The supernatant was decanted and 500 uL to 1 mL of RLT plus buffer (Qiagen DNA/RNA All-Prep Kit) was added. Cell pellets were resuspended by vortexing and a needle/syringe was used to aid in cell lysis. The DNA/RNA All-Prep Kit (Qiagen) was used to extract DNA following manufacturer's instructions. DNA concentration was quantified using the Qubit dsDNA High Sensitivity Assay Kit (Life Technologies, Carlsbad, CA, United States).

To generate 16S rRNA gene amplicons, purified DNA was amplified using 16S rRNA gene primers specific to the V4 region (515F/806R) (Caporaso J G et al., 2011, Proceedings of the National Academy of Sciences 108:4516-4522, the entirety of which is incorporated by reference herein). Each sample was pooled in 2 technical replicates to ensure enough PCR product was generated. In order to ensure no contamination occurred, negative PCR controls were periodically amplified and quantified. After amplification, PCR products were purified using SPRIselect beads (Beckman Coulter, Pasadena, CA), according to the manufacturer's protocol. A 2 nM amplicon library was generated by pooling and diluting PCR products. The pooled amplicon library was denatured with fresh 0.2 N sodium hydroxide for five minutes. Libraries were further processed according to the manufacturer's protocol and sequenced on an Illumina MiSeq sequencer using a 300-cycle V2 Nano kit (Illumina, San Diego, CA).

The 16S rRNA gene sequences were analyzed using QIIME2 version 2021.4. All 16S sequences were imported as EMPSingleEndSequences and subsequently demultiplexed using the demux emp-single command. DADA2 (Callahan B J et al., 2016, Nature Methods 13:581-583, the entirety of which is incorporated by reference herein) was used for quality control and to filter out any chimeric sequences from the demultiplexed data. The truncation length for these sequences occurred at 250 bases. In order to determine the taxonomic composition of these samples, the classify-sklearn command was used (Pedregosa F. et al., 2011, Journal of Machine Learning Research 12:2825-2830, the entirety of which is incorporated by reference herein) in conjunction with a pre-trained Naive Bayes classifier trained on Silva 132 99% OTUs (Quast C. et al., 2012, Nucleic Acids Research 41:D590-D596, Yilmaz P. et al., 2014, Nucleic Acids Res 42:D643-8, the entirety of both incorporated by reference herein) from the 515F/806R region to classify representative sequences from DADA2.

Quantification of 16S Gene Copies Using Digital Droplet PCR (ddPCR)

Samples were analyzed using digital droplet PCR technology to determine the absolute quantification of 16S gene concentration in copies per microliter (μL). A mastermix was created using 8.6 μL of PCR grade H2O, 11.0 μL of QX200 ddPCR EvaGreen Supermix (BioRad), 0.2 μL of both a 16S rRNA gene forward and reverse primer per sample Maeda H. et al., 2003, FEMS Immunology and Medical Microbiology 39:81-86; Tinker K. et al., Frontiers in Microbiology 11, the entirety of both incorporated by reference herein. 20 μL of the mastermix was aliquoted into an 8-well PCR strip tube where 2 μL of DNA was added. Sample DNA was diluted to a concentration of 10-2 to prevent oversaturation in the ddPCR. One negative control was utilized per run, with UV-crosslinked PCR H₂O used as the sample. PCR tube strips were carefully capped and centrifuged to mix the mastermix and ensure all the liquid was captured at the bottom of the tube. 20 μL of this mastermix was subsequently loaded into a DG8 cartridge, and 70 μL of Droplet Generation Oil for EvaGreen was added into the oil well of the cartridge. The cartridge was fitted with a single-use gasket and loaded onto a QX200 droplet generator. Following droplet generation, a multichannel pipette was used to pipette 40 μL of the sample into a semi-skirted 96-well plate (BioRad). After all samples were transferred, a pierceable foil was placed onto the 96-well plate and heated at 180° C. for 5 seconds to heat-seal the plate. The 96-well plate was loaded onto a C1000 Touch Deep-Well Thermal Cycler and run under the following protocol, adapted from the manufacturer: 95° C. for 5 min, [95° C. for 30 s, 60° C. for 60 s]×40, 4° C. for 5 min, 90° C. for 5 min. The plate was then loaded into the QX200 Droplet Reader and analyzed using BioRad's QX Manager Standard Edition software.

### Metagenome Sequencing, Read Processing, Assembly, and Analysis

Shotgun metagenome sequencing was performed on an Illumina MiSeq sequencer using a MiSeq Reagent kit v3 (600-cycle). Genomic DNA was extracted with a DNA/RNA AllPrep kit (Qiagen), according to the manufacturer's instructions. DNA concentration was quantified using the Qubit dsDNA High Sensitivity Assay Kit (Life Technologies, Carlsbad, CA, United States). DNA quality was determined with a bioanalyzer (Agilent, Santa Clara, CA, United States). DNA libraries were prepared using the Nextera XT DNA Library Preparation Kit according to manufacturer's instructions (Illumina, San Diego, CA). Raw paired-end metagenome reads were uploaded to the kBase webserver. Raw read quality was assessed with FastQC v0.11.9. Reads were trimmed with Trimmomatic v0.36 (Sliding window size=4, sliding window minimum quality=15) (Wu Y-W. et al., 2015, Bioinformatics 32:605-607 the entirety of which incorporated by reference herein). Trimmed read pairs were again assessed for quality (FastQC), assembled with metaSPAdes v3.15.3 (minimum contig length=300≤2000) (Nurk S et al., 2017, Genome Res 27:824-834, the entirety of which is incorporated by reference herein), and the resultant contigs were binned using MaxBin2 v2.2.4 (probability threshold=0.8, marker set=107 bacterial marker gene set, minimum contig length=1000) (Wu Y-W. et al., 2015, Bioinformatics 32:605-607, the entirety of which is incorporated by reference herein). Metagenome-assembled genome (MAG) quality was assessed with QUAST (Mikheenko A. et al., 2018, Bioinformatics 34:i142-i150, the entirety of which is incorporated by reference herein) and CheckM v1.0.18 (default parameters) (Parks D. et al., 2015, Genome Res 25:1043-55, the entirety of which is incorporated by reference herein). Objective taxonomic assignment of each MAG was performed with GTDB-tk v1.7.0 (Parks D H. et al., 2018, Nature Biotechnology 36:996-1004; Chaumeil P-A. et al., 2019, Bioinformatics 36:1925-1927; Parks D H. et al., 2020, Nature Biotechnology 38:1079-1086; Matsen F A. et al., 2010, BMC Bioinformatics 11:538; Hyatt D. et al., 2010, BMC Bioinformatics 11:119; Price M N. et al., 2010, PLOS One 5:e9490; Price M N. Et al., 2010, PLOS One 5:e9490; Eddy S R et al., 2011, PLOS Comput Biol 7:e1002195; Jain C. et al., 2018, Nature Communications 9:5114, the entirety of all nine are incorporated by reference herein).

### Coal Seam Produced Water (CSPW) Microbiome

Coal seams are a unique subsurface environment that has previously been found to harbor a diverse population of microorganisms capable of utilizing various carbon compounds, coal intermediates, and fermentation products to produce methane

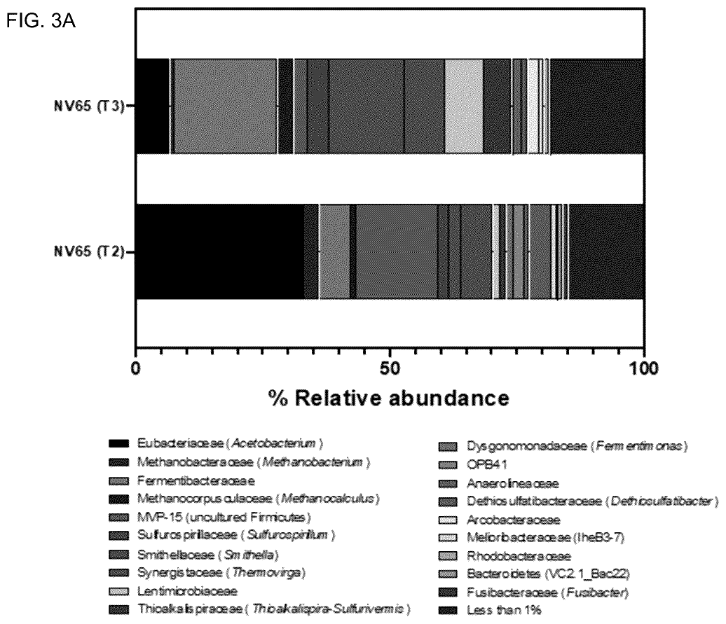

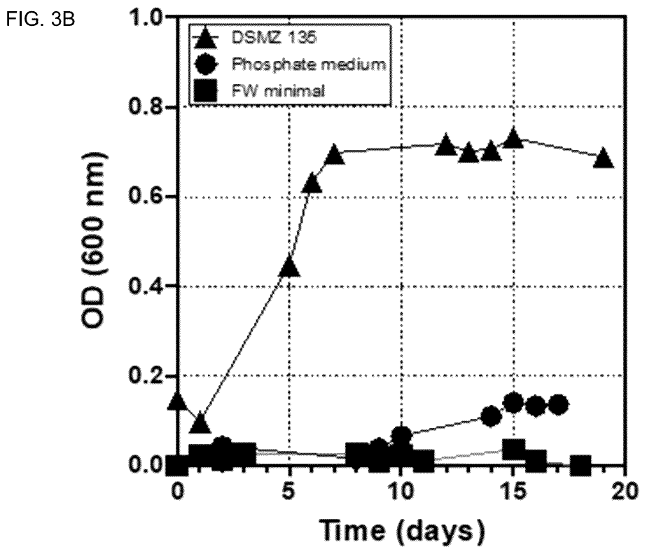

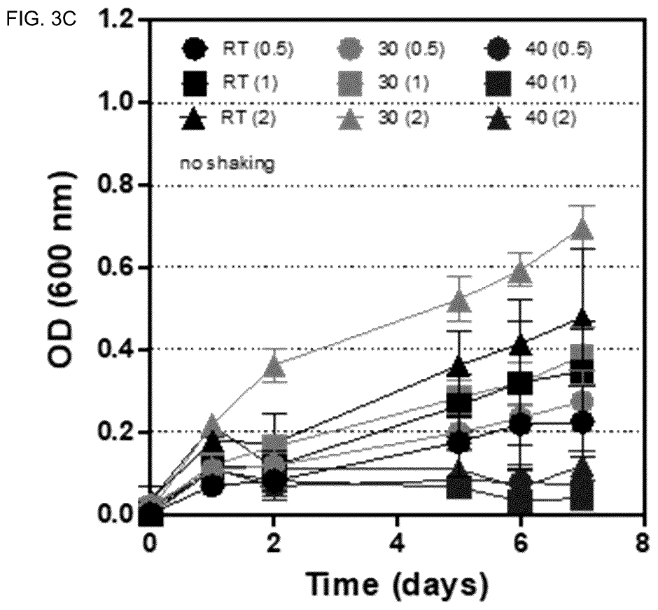

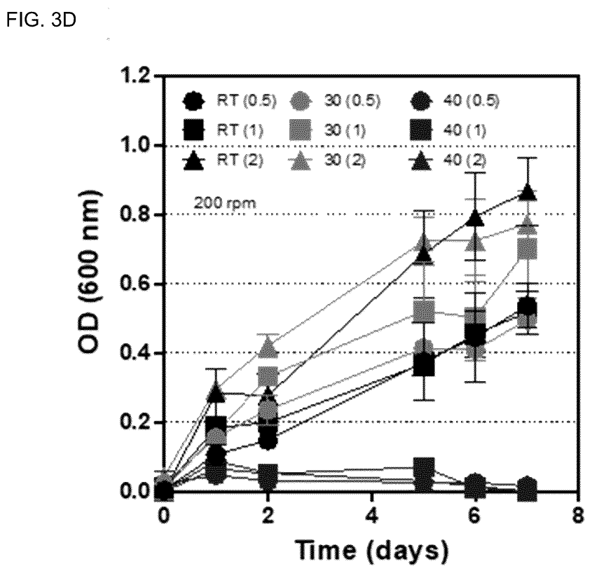

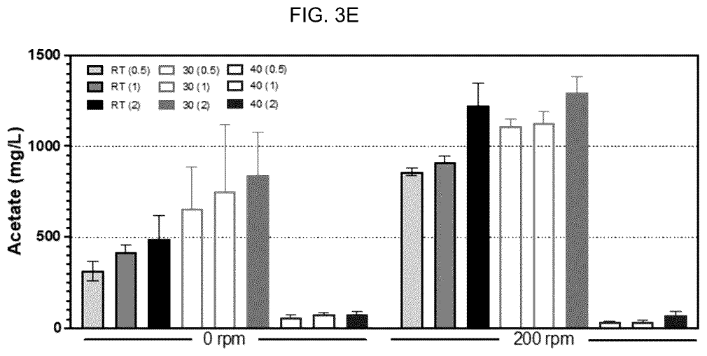

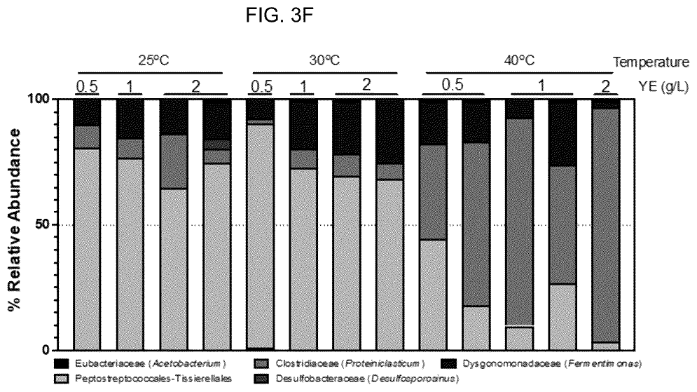

Produced water from the Appalachian Basin was sampled from a coal seam well in Southwestern Pennsylvania, USA. And was comprised of a variety of taxa, including Eubacteriaceae, Methanobacteraceae, Smithellaceae, Fermentibacteraceae, Methanocorpusculaceae, Sulfurospirillaceae, Synergistaceae, Lentimicrobiaceae, Thioalkalispiraceae, Dysgonomonadaceae, and Dethiosulfatibacteraceae (FIG. 3A). Initial enrichment of coal seam produced water microbiome NV65.

Unfiltered coal seam produced water was inoculated into three different media with H₂:CO₂ (80:20) gas to enrich for autotrophic microorganisms. Freshwater ‘FW’ medium and phosphate-buffered medium were utilized to establish a stable microbial community for CO₂ conversion in microbial electrosynthesis systems (MES) (Marshall C W et al., 2013, Environmental Science & Technology 47:6023-6029; LaBelle E V et al., 2014, PLOS ONE 9:e109935), while DSMZ 135 is the standard medium recommended for culturing Acetobacterium, a taxon containing autotrophic acetogens. Growth was only observed for DSMZ 135 medium (FIG. 3B), while little to no growth was observed for cultures inoculated in ‘FW’ minimal medium or the phosphate-buffered minimal medium. Based upon these initial findings, the modified DSMZ 135 medium was used in all subsequent experiments.

### Temperature, Agitation, and Yeast Extract Increase Growth and Acetate Production

Many variables affect product selectivity and yield in microbial gas fermentation, including gas composition, medium composition, agitation, pH, and temperature. To test how agitation, temperature, and medium composition affect growth and product formation of the enriched coal seam biocatalyst, experiments were executed over a 7-day period with and without agitation, and with varying temperature (room temperature (˜25° C.), 30° C., and 40° C.), and yeast extract (YE) (0.5, 1, and 2 g/L) (FIGS. 3C-3F). Agitation had a positive effect on growth and acetate titers, as incubations that were agitated at 200 rpm grew faster, had higher final ODs, and produced more acetate than stagnant cultures (no shaking) (FIGS. 3C-3E). The effect of agitation was most prominent in the room temperature incubations, with shaking cultures producing 2.2-2.7× more acetate than stagnant cultures. Agitation had less of an effect at 30° C., with acetate titers from shaking cultures only 1.5-1.7× greater than stagnant cultures.

Next, biocatalyst performance at various temperatures was examined. Incubations at 30° C. yielded the fastest growth and highest acetate tiers, followed by 25° C., while incubations at 40° C. exhibited the least amount of growth and produced small amounts of acetate (37-79 mg/L) (FIGS. 3C-3E). Yeast extract (YE) is used as a medium supplement for cell cultures, supplying vitamins, amino acids, nitrogen, and carbon (Leclerc et al., 1998; Sommer, 1998) FIGS. 3C-3E. Overall, increasing concentrations of yeast extract yielded increasing ODs and acetate titers. For example, at 25° C. stagnant cultures with 0.5, 1, or 2 g/L YE produced 314, 418, and 489 mg/L acetate, respectively. The 30° C. stagnant cultures performed better than the 25° C. stagnant cultures at similar YE concentrations, with acetate titers ranging from 654-850 mg/L (FIGS. 3A-3E). With shaking, the 25° C. cultures with 0.5, 1, or 2 g/L YE produced 859, 913, and 1,223 mg/L acetate, respectively. Acetate titers for agitated cultures at 30° C. increased to 1,114, 1,129, and 1,299 mg/L, with the addition of 0.5, 1, and 2 g/L YE, respectively. Under all conditions at 40° C., acetate titers were <100 mg/L, though the highest increase in acetate titers was observed between incubations with 0.5 g/L and 2 g/L YE.

To understand what types of microorganisms were present in enrichment cultures, how experimental parameters may affect the microbial community, and how the microbial community composition may relate to product formation, amplicon sequencing of the 16S rRNA gene from samples at all temperatures and yeast extract concentrations were performed (FIG. 3E). All samples were comprised of the following taxa (>1% relative abundance): Peptostreptococcales-Tissierellales, Clostridiaceae (Proteiniclasticum), Desulfobacteraceae (Desulfosporosinus), Dysgonomonadaceae (Fermentimonas), and Eubacteriaceae (Acetobacterium). Peptostreptococcales-Tissierellales was resolved to the order level and had the highest relative abundances at all yeast extract concentrations at 25° C. and 30° C., with relative abundances ranging from 64.9 to 89.6% (FIGS. 3A-3E). The next most abundant taxa were resolved to the genus level and included Fermentimonas, ranging from 7.6 to 25.3%, followed by Proteiniclasticum (1.7-21.4%), Desulfosporosinus (0.05-4.14%), and Acetobacterium (0.4-1.0%). The taxonomic profile at 40° C. was markedly different than 25° C. and 30° C., with Proteiniclasticum having the highest representation (38.4-93.7%), followed by Peptostreptococcales-Tissierellales (3.3-43.9%), Fermentimonas (2.9-25.8%), and Acetobacterium (0.11-0.32%). This may be a result of lower optical densities (<0.1), and 16S rRNA gene copies per mL (3.0×10⁵ to 6.8×10⁷), compared to 25° C. and 30° C. (OD range=0.2-0.9; 16S rRNA gene copies per mL range=1.0×10⁷−2.4×10⁸).

Enhanced Growth and Product Formation with Bioreactor Scale-Up

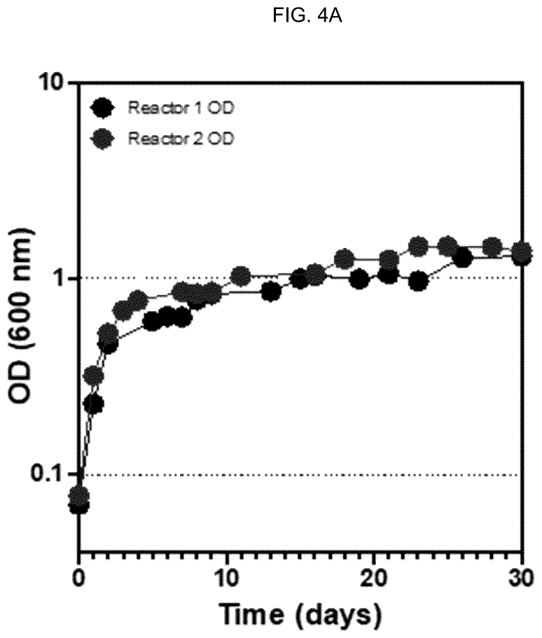

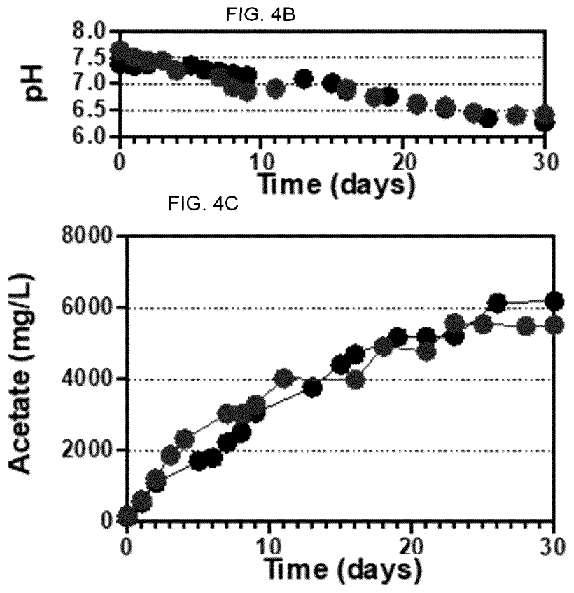

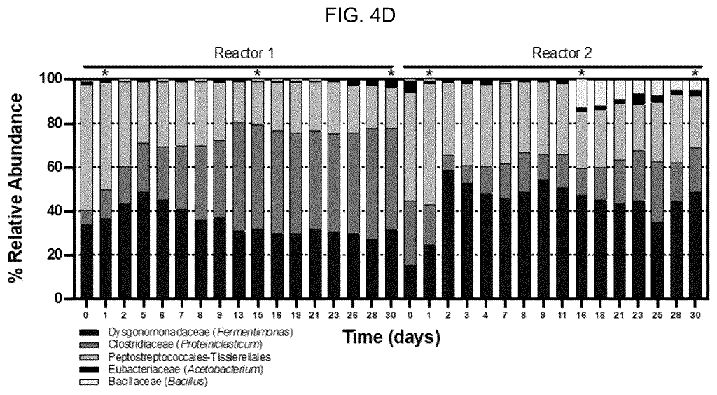

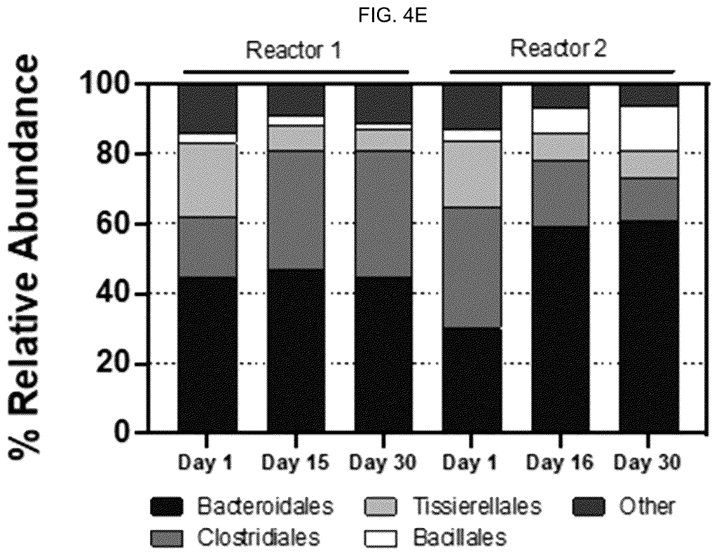

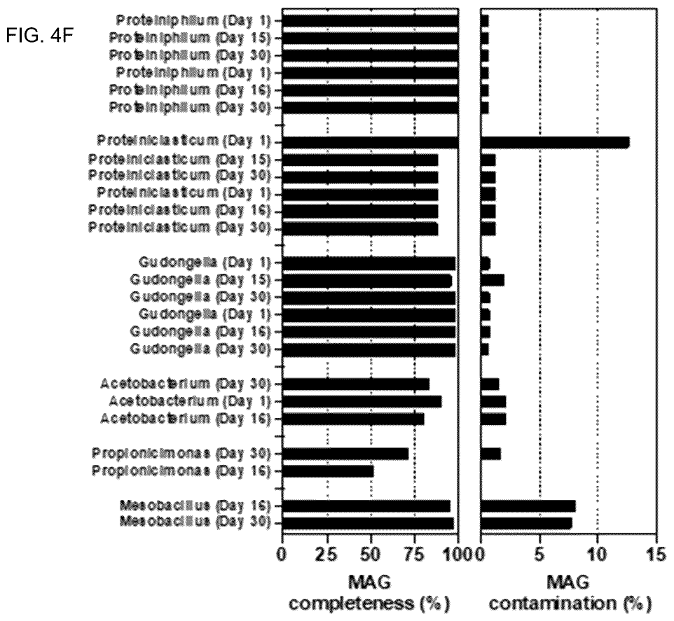

As a first step towards a higher bioindustrial manufacturing readiness level, batch reactors were scaled up to 1 L (500 mL working volume, 500 mL gas headspace). Duplicate reactors were operational for 30 days in a semi-batch mode with no pH control, where the gas headspace was replenished with H₂:CO₂ (80:20) at each timepoint (FIGS. 4A-4F). Biocatalyst growth was fastest over the first few days and slowed for the duration of the experiment (FIG. 4A). Initial cell concentrations ranged from 4.52-9.92×10⁶ 16S rRNA gene copies per mL, with cultures reaching a maximal concentration of 2.29×10⁸ copies per mL. The pH remained stable over the first 5 days (˜7.3-7.5), but from day 5 to day 30, slowly decreased to 6.3 (FIG. 4B). Over the first 7 days, the 1 L reactors produced 2,227 and 3,034 mg/L of acetate (FIG. 4C), compared to 1,000-1,600 mg/L of acetate for small batch reactors (10 mL working volume). After 30 days of operation, acetate titers for 1 L reactors reached 5,523 and 6,194 mg/L. Production rates were highest over the first four days of operation (0.582 g/L/day), slowed to an average of 0.366 g/L/day over the first eleven days, and 0.184 g/L/day over 30 days.

Throughout the experiment a negative pressure was observed when sampling, suggesting utilization of the supplied anaerobic gas mixture (H₂:CO₂). Under experimental conditions available gaseous CO₂ may be limiting, therefore, biocatalyst growth and acetate production may be enhanced with a continuous supply of gas. Furthermore, over the course of the experiment the medium is being depleted of essential nutrients for efficient bioconversion, concomitant with a decrease in pH due to the utilization of sodium bicarbonate and/or production of acetic acid. As such, a constant supply of fresh medium may help overcome these limitations and enhance production rates. At present, bioreactors are optimized for semi-high throughput for testing various experimental parameters. To enhance biocatalyst conversion rates, alternative reactors may be employed, such as a continuous stirred tank reactor (CSTR) or trickle bed reactor, which allow for constant mixing, gas sparging, and pH control. Future work will examine alternative reactor configurations and feeding strategies to optimize growth and CO₂ conversion.

Taxonomy of the enriched microbial community was monitored over the course of the experiment and as observed in earlier experiments, the microbial community composition of the biocatalyst from 1 L reactors was comprised of Fermentimonas, Proteiniclasticum, Peptostreptococcales-Tissierellales, Acetobacterium, and Bacillus (FIG. 4D). At the start of the experiment, Peptostreptococcales-Tissierellales was the most abundant (relative abundance ˜60%), followed by Fermentimonas and Proteiniclasticum. Fermentimonas increased over the first few days (from ˜15-30% to ˜45-60%) and decreased over the remainder of the experiment to a final relative abundance of ˜30-45%. Proteiniclasticum increased over the course of the experiment and remained relatively stable from day 2 onward. Fermentimonas was slightly elevated in reactor 2 (˜40-60% relative abundance), compared to reactor 1 (˜30-50%). Conversely, the relative abundance of Proteiniclasticum was elevated in reactor 1 (˜20-50%) compared to reactor 2 (˜5-10%). Interestingly, Bacillus was present in both reactors (<1% relative abundance) but in reactor 2 Bacillus increased to 12% relative abundance, slowly decreasing to 4.7% at the end of the experiment (FIG. 4A-4F). Despite variations in relative abundances between reactors, no major differences were observed between changes in growth, pH, and acetate production for each reactor, suggesting potential metabolic overlap between microbial community members of the enriched biocatalyst.

Metagenome sequencing was performed on samples at the beginning (Day 1), middle (Day 15 or 16), and end (Day 30) of the 1 L experiments. Taxonomic identification of unassembled metagenome reads revealed similar relative abundances to what was observed with 16S rRNA gene amplicon sequencing (FIG. 4E). Metagenome-assembled genomes (MAGs) were generated via assembly and binning of metagenome reads. MAGs were classified into six different bacterial genera, including four Firmicutes (Gudongella, Proteiniclasticum, Acetobacterium, and Mesobacillus), one Bacteroidota (Proteiniphilum), and one Actinobacteriota (Propionicimonas). Based upon the presence of single copy marker genes, the MAGs were estimated to be between ˜71% (Propionicimonas) to 100% complete (Proteiniclasticum), with the majority of MAGs >88% complete, and <2% contamination (FIG. 4F). Approximately 98% of the paired metagenome reads mapped to the metagenome-assembled genomes.

Formatotrophic Growth of a Biocatalyst Enriched from a Coal Seam Produced Water (CSPW) Microbiome

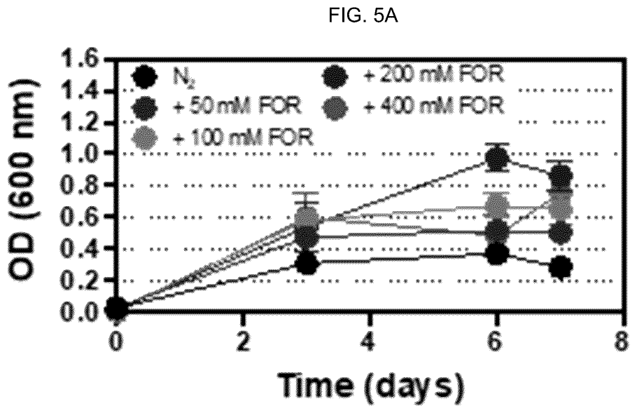

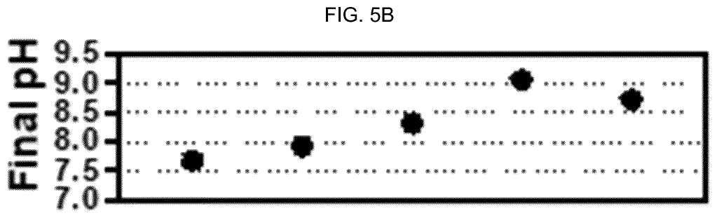

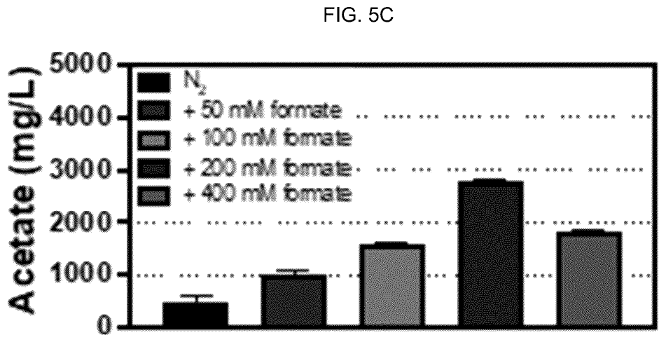

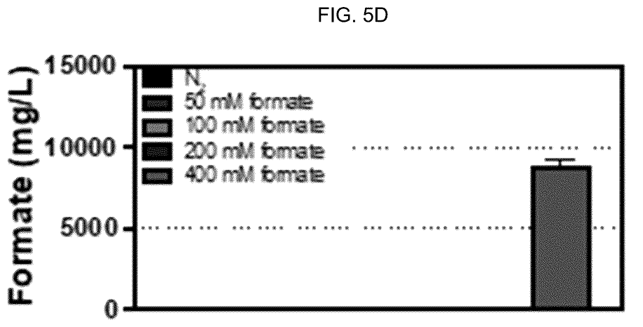

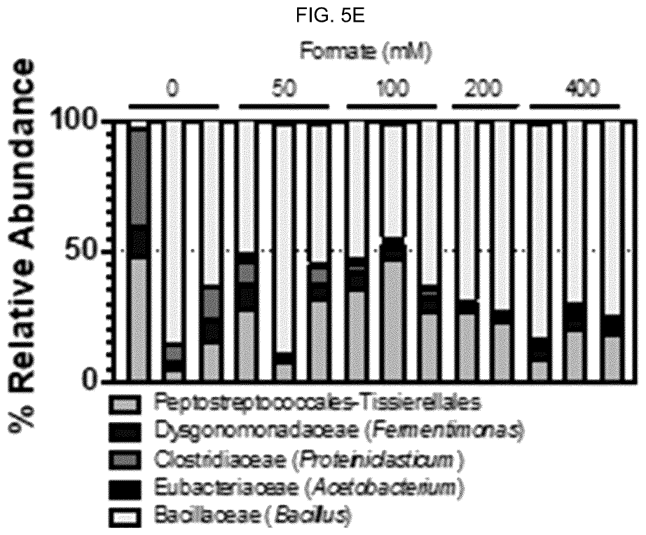

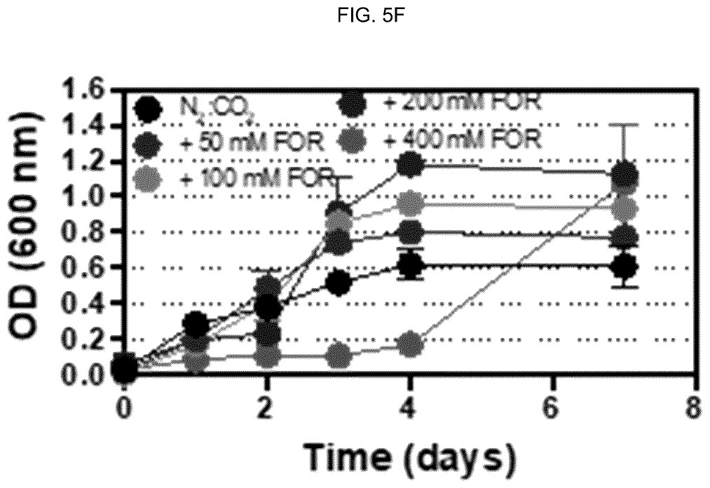

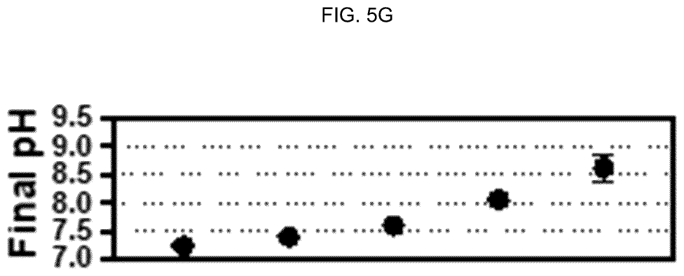

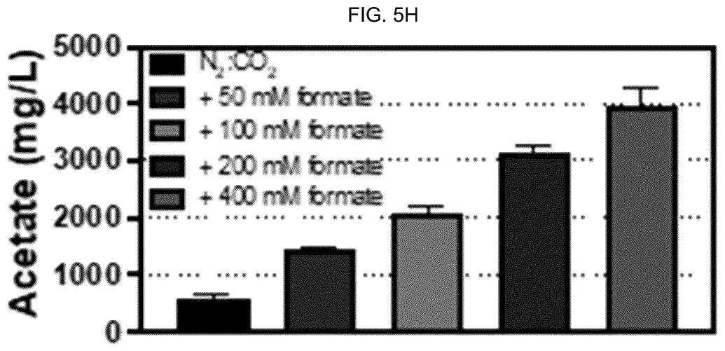

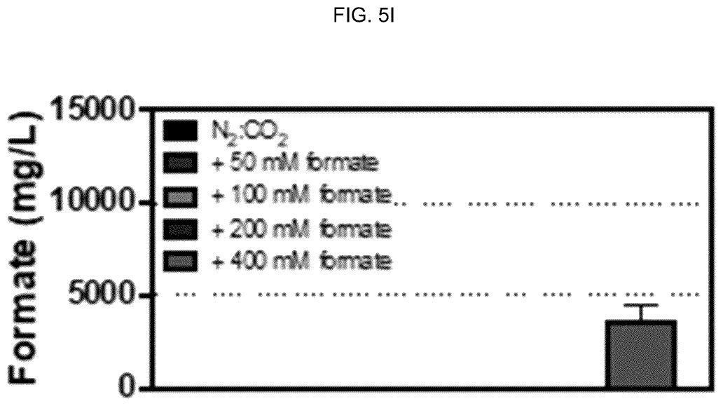

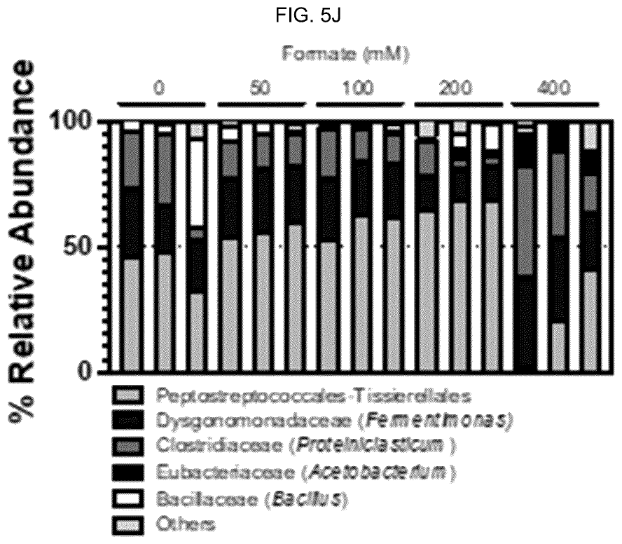

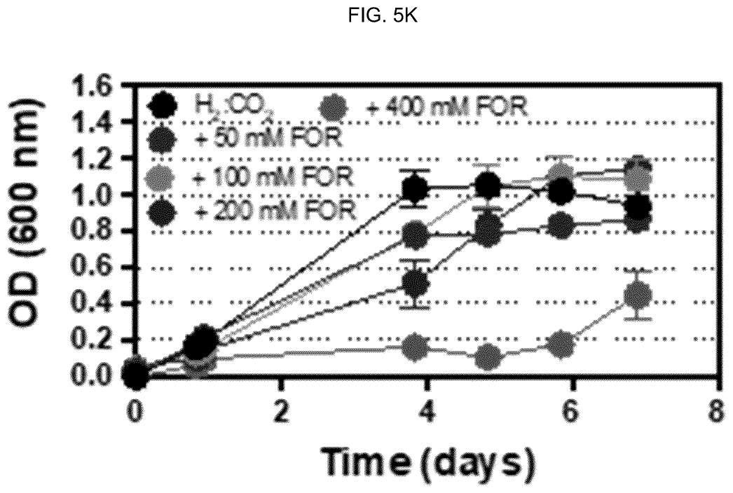

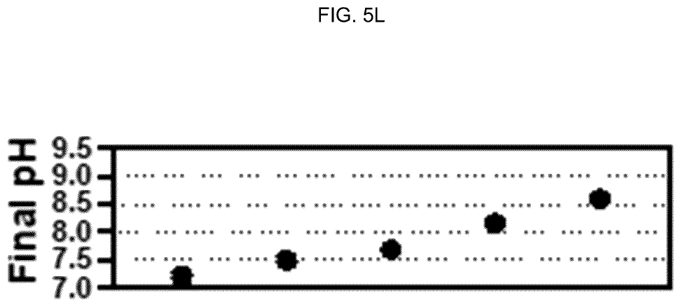

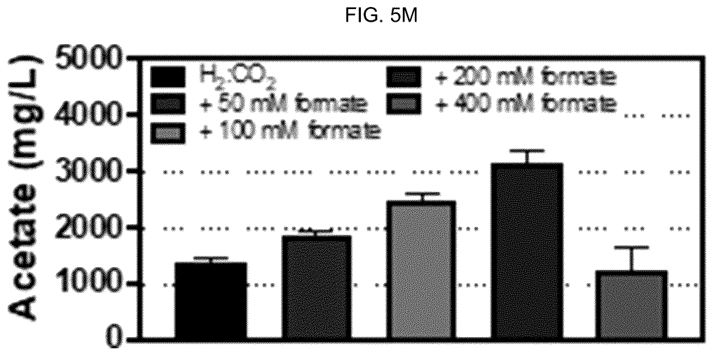

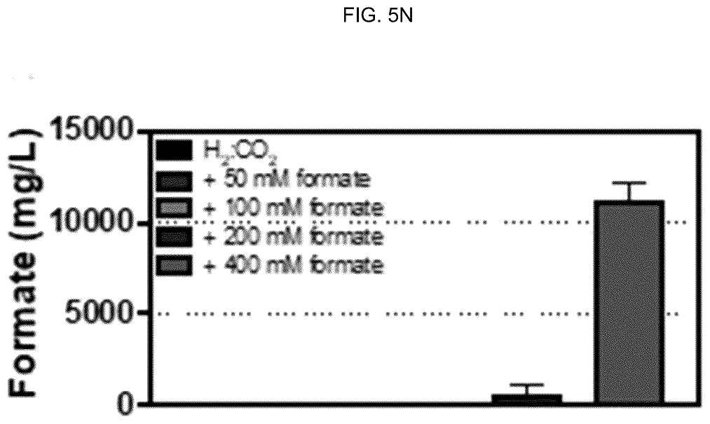

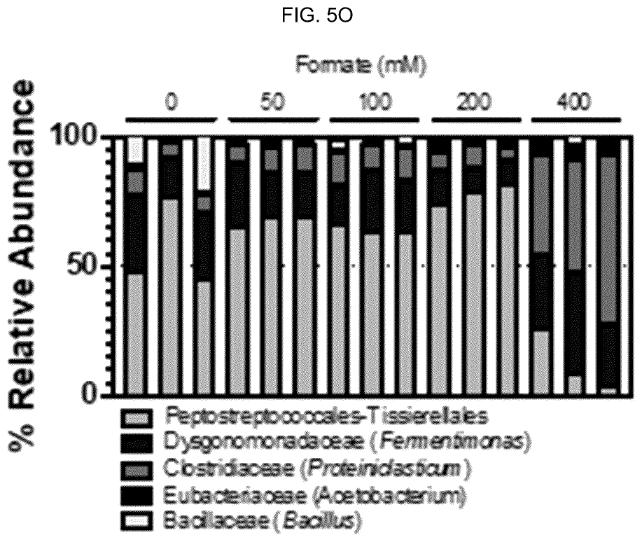

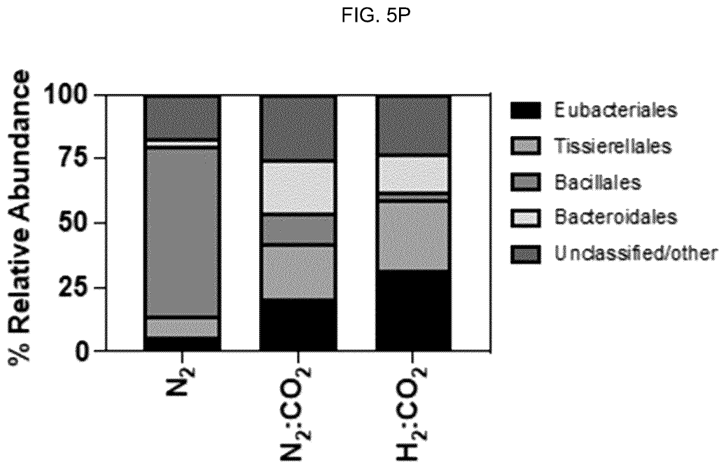

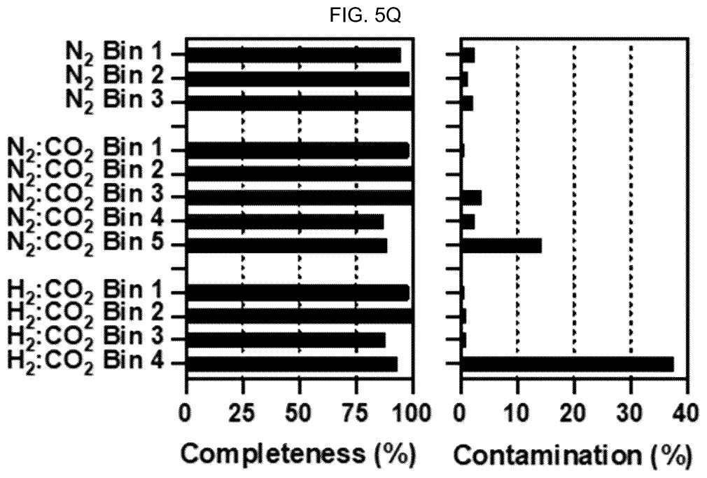

To examine the potential for formate utilization by the CSPW biocatalyst (invented biocatalyst), a series of batch tests were performed under anaerobic conditions (N₂, N₂:CO₂, or H₂:CO₂) with varying formate. Under an N₂ atmosphere (unitrophic regime), the CSPW biocatalyst grew to an OD₆₀₀ of 0.4 and had a final pH of 7.68. Acetate was the main short chain organic acid produced. With addition of increasing formate (50-400 mM), the biocatalyst exhibited increased growth (FIG. 5A), increased final pH (FIG. 5B) and produced increasing amounts of acetate (FIG. 5C). Acetate titers were highest (˜3,000 mg/L) at a formate concentration of 200 mM. At 400 mM formate, acetate titers decreased, and some unutilized formate was still present (FIG. 5D). Examination of the microbial community via 16S rRNA gene amplicon sequencing revealed five main taxa were present, including Bacillaceae (Bacillus), Peptostreptococcales-Tissierellales, Proteiniclasticum, Fermentimonas, and Acetobacterium (FIG. 5E).

Formatotrophic growth with N₂:CO₂ (mixotrophy) was observed with a range of formate, with cultures reaching optical densities of 0.61 to 1.13 after 7 days of incubation (FIG. 5F). Increasing formate concentrations led to a longer lag phase in growth, the longest of which was ˜4 days (400 mM formate) but did not affect final OD₆₀₀ values or acetate titers. Final pH values of culture supernatant ranged from 7.23 (0 mM formate) to 8.63 (400 mM formate) (FIG. 5G). A similar increase in pH was observed with the acetogen Acetobacterium woodii grown on formate. Increased formate concentrations yielded larger acetate titers, ranging from 598 mg/L (no formate) to 3,958 mg/L acetate (400 mM formate), a 6.6× increase (FIG. 5H). Almost all formate (˜99%) was utilized in the 50-200 mM formate incubations, while ˜80% was utilized for the 400 mM formate incubations (FIG. 5I). The microbial community structure was markedly different from N₂+formate incubations, with Peptostreptococcales-Tissierellales having ≥50% relative abundance in the 0-200 mM formate incubations (FIG. 5J). At 400 mM formate, Fermentimonas and Proteiniclasticum were the predominant taxa, followed by Peptostreptococcales-Tissierellales, Acetobacterium, and Bacillus.

Next, the co-utilization of H₂:CO₂ and formate was examined. Over the first four days, H₂:CO₂ cultures without formate had the fastest growth (FIG. 5K). At the conclusion of the experiment on day 7, the 100 and 200 mM formate incubations had the highest optical densities, suggesting formate enhances growth, albeit with slower growth rates/longer lag phase. Likewise, formate addition enhanced acetate titers, with acetate production increasing from 1,363 mg/L (H₂:CO₂, no formate) to 3,132 mg/L (H₂:CO₂, 200 mM formate), a 2.3× increase (FIG. 5M). At 400 mM formate, cultures had the longest lag phase and produced the least amount of acetate. Of the initial formate provided, ˜99% was utilized with 50 mM and 100 mM formate, 94% was utilized with 200 mM formate, and 39% was utilized with 400 mM formate (FIG. 5N). The final pH was 7.21 for cultures without addition of formate, and incremental addition of formate yielded increasing final pH values, with the pH of 400 mM incubations increasing to 8.59 (FIG. 5O). Based on 16S rRNA gene sequencing the CSPW biocatalyst was predominantly comprised of three main taxa, including Peptostreptococcales-Tissierellales (46-82% relative abundance) at 0-200 mM formate, followed by Fermentimonas (9.2-30%) and Proteiniclasticum (9.4-12.7%). The community structure was decidedly different at 400 mM formate, with Proteiniclasticum (38.6-66.4%) and Fermentimonas (23.5-40.1%) having the highest representation, followed by Peptostreptococcales-Tissierellales (4.5-26.3%) (FIG. 5P). Formatrophic growth and organic acid production with various pure and mixed gasses

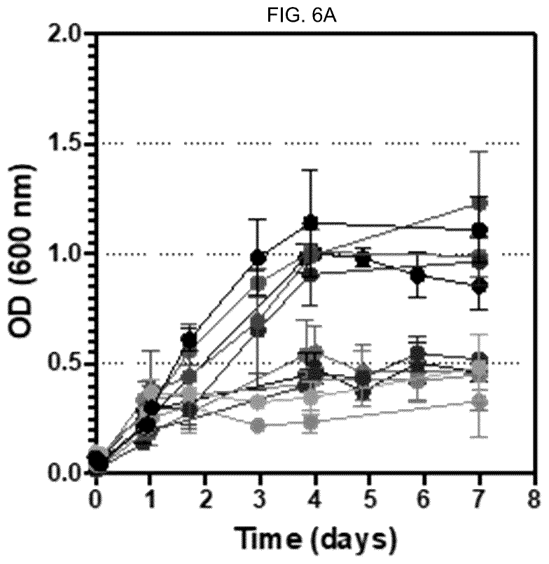

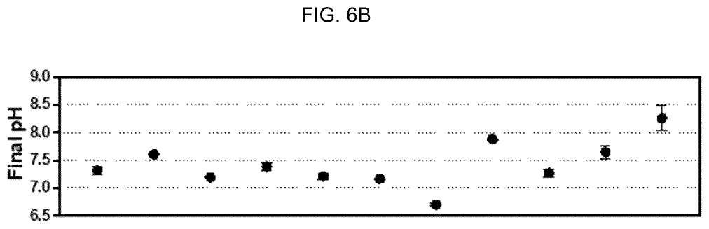

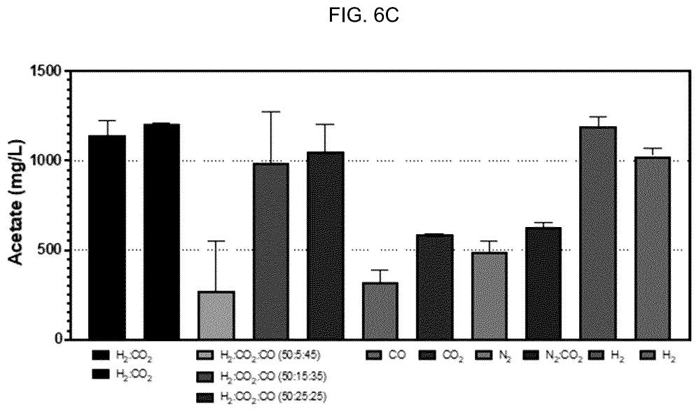

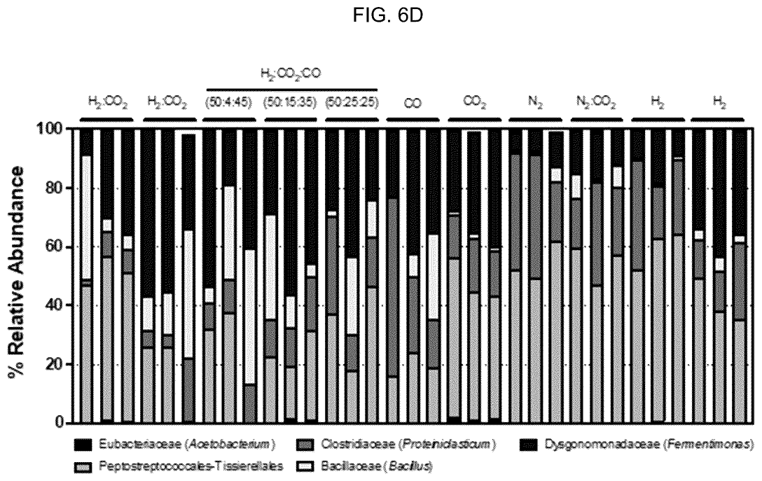

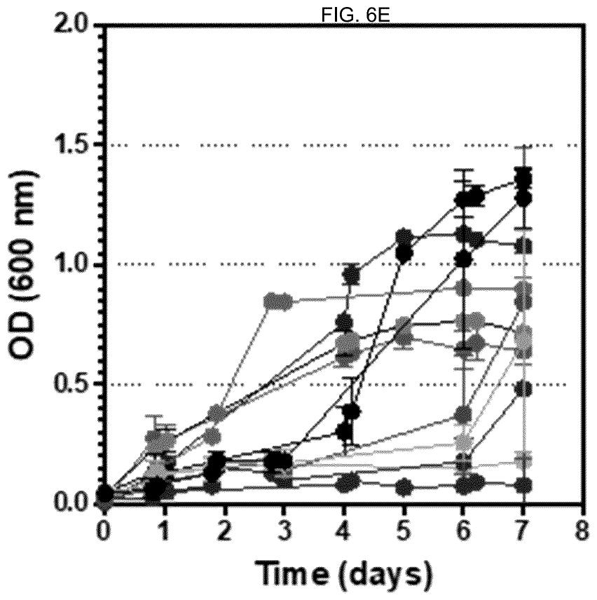

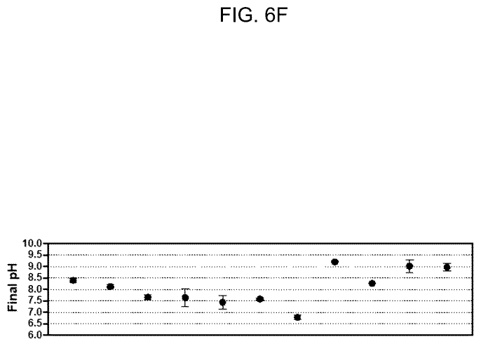

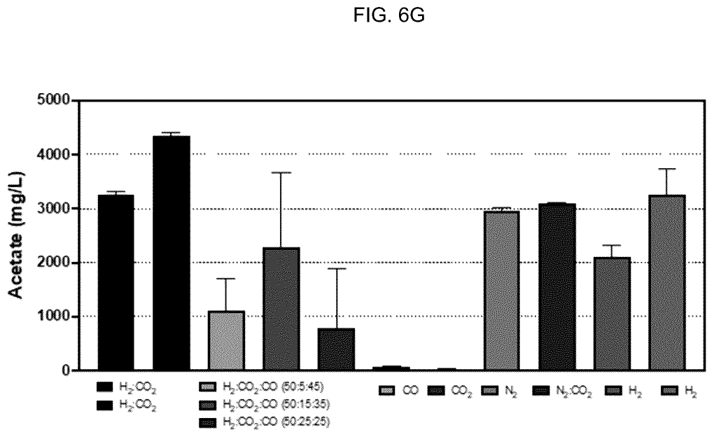

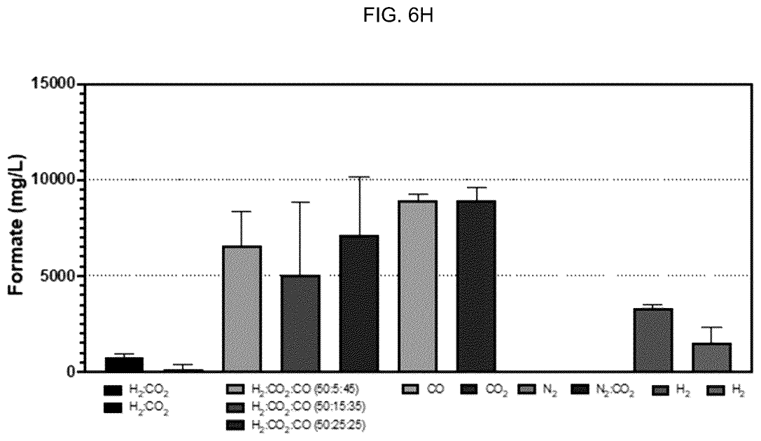

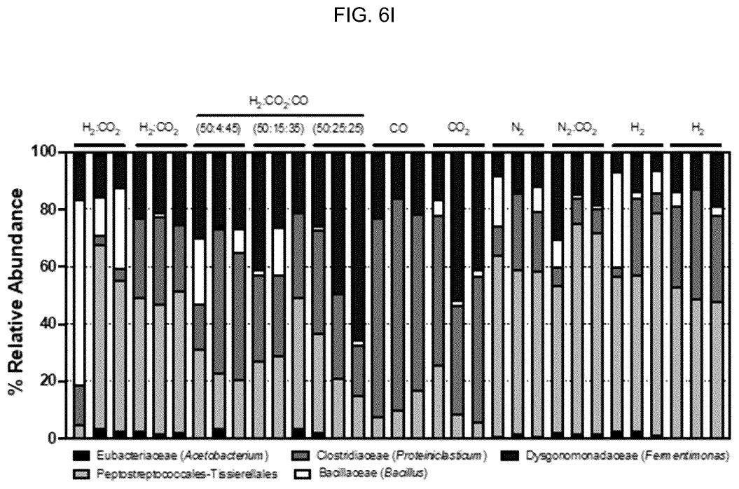

Waste CO₂ streams (such as syngas) often contain other gaseous components such as H₂ and CO. To examine the ability of using alternative gas substrates and expanding the utility of CSPW microbiome biocatalysis, growth and organic acid production was examined using various pure and mixed gasses, including H₂:CO₂:CO (50:5:45), H₂:CO₂:CO (50:15:35), H₂:CO₂:CO (50:25:25), 100% CO₂, or 100% H₂ (FIGS. 6A-61). In the absence of formate, growth was most robust with H₂:CO₂, H₂, H₂:CO₂:CO (50:15:35), or H₂:CO₂:CO (50:25:25), reaching a max optical densities of 1 to 1.25, followed by CO₂, H₂:CO₂:CO (50:5:45), N₂, N₂:CO₂, and CO, with optical densities ranging from 0.45 to 0.52 (FIG. 6A). Final pH values at the conclusion of each experiment were varied, depending upon the supplied gas. For example, H₂:CO₂ enrichments had a final pH of 7.32, while CO₂ had a lower pH (6.70), and H₂ had a higher final pH (7.88 and 8.26, respectively) (FIG. 6B). Acetate production was observed with all gasses tested, with H₂:CO₂ enrichments producing the most (1,203 mg/L), followed by H₂ (1,192 mg/L), H₂:CO₂:CO (50:25:25) (1,050 mg/L), H₂ (1,027 mg/L), H₂:CO₂:CO (50:15:35) (985 mg/L), N₂:CO₂ (626 mg/L), CO₂ (587 mg/L), N₂ (488 mg/L), CO (319 mg/L), and H₂:CO₂:CO (50:25:25) (272 mg/L) (FIG. 6C).

Depending upon the supplied gas, the dominant taxa of the CSPW microbiome (invented biocatalyst) was varied (FIG. 6D). Peptostreptococcales-Tissierellales had the highest relative abundances and were predominant in samples containing H₂:CO₂, CO₂, N₂, N₂:CO₂, or H₂. Conversely, experiments containing any amount of CO had markedly different community structures, including a higher relative proportion of Bacillaceae and lower relative abundance of Peptostreptococcales-Tissierellales. In all cases, irrespective of gas composition, the main taxa were still present, albeit at varying relative abundances.

Next, utilization of formate with various pure and mixed gasses was examined. Cultures grown on H₂:CO₂ and formate (200 mM) exhibited the highest optical densities (1.36 OD₆₀₀), followed by N₂:CO₂, H₂, N₂, H₂:CO₂:CO (50:15:35), H₂:CO₂:CO (50:5:45), H₂:CO₂:CO (50:25:25), CO, and CO₂ (FIG. 6E). Growth was inhibited over the first four days for H₂:CO₂:formate cultures, followed by a sharp increase between day 4 and day 5. Medium pH values ranged from 6.78 (CO₂) to 9.20 (N₂), with most samples with pH in the range of 7.5 to 8.5 (FIG. 6F). Acetate production above 500 mg/L was observed in incubations with all gasses, except for 100% CO, and 100% CO₂, and acetate titers ranged from 780 mg/L [H₂:CO₂:CO (50:25:25)], to 4,337 mg/L (H₂:CO₂) (FIG. 6G). Unutilized formate was present in all incubations, though higher acetate production corresponded to lower amounts of remaining formate, suggesting formate conversion to acetate (FIG. 6H).

The CSPW biocatalyst (invented biocatalyst) grown with formate and various pure and mixed gasses contained the same taxa as previously described but with varying relative abundances relative to the gas atmosphere provided (FIG. 6I). Specifically, Peptostreptococcales-Tissierellales was predominant (>50% relative abundance) in incubations containing formate and H₂:CO₂, N₂, N₂:CO₂, or H₂, while Clostridiaceae (Proteiniclasticum), and Dysgonomonadaceae (Fermentimonas) had higher relative abundances in incubations with 100% CO, the three H₂:CO₂:CO mixtures, and 100% CO₂. Overall, cultures producing the most acetate contained the highest relative amounts of Peptostreptococcales-Tissierellales.

Bacterial MAGs Reassembled from an Enriched Coal Seam Produced Water Microbiome

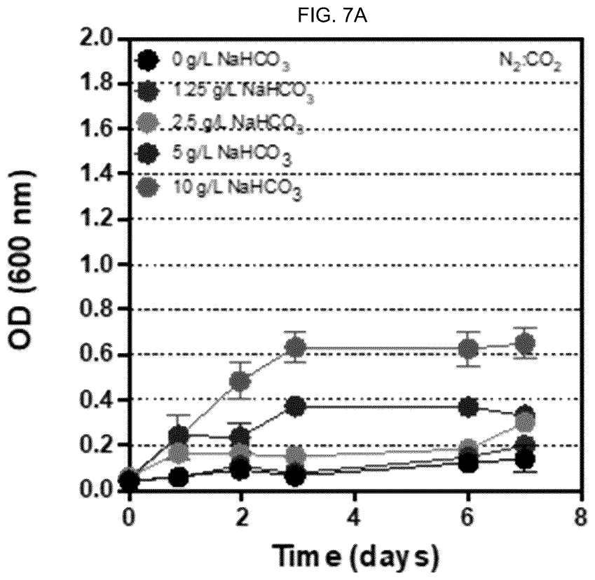

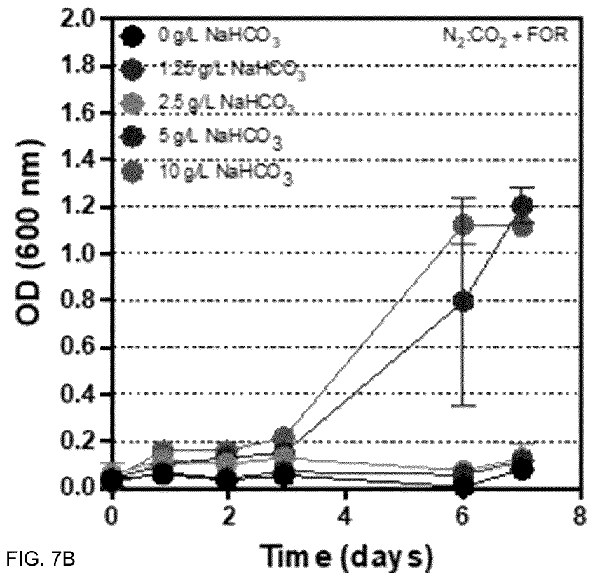

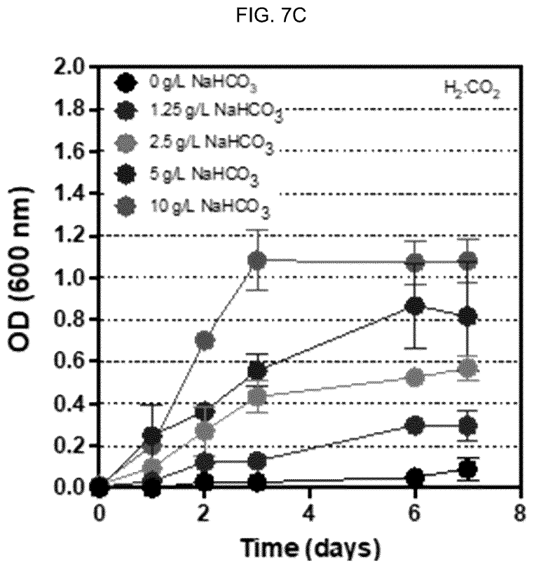

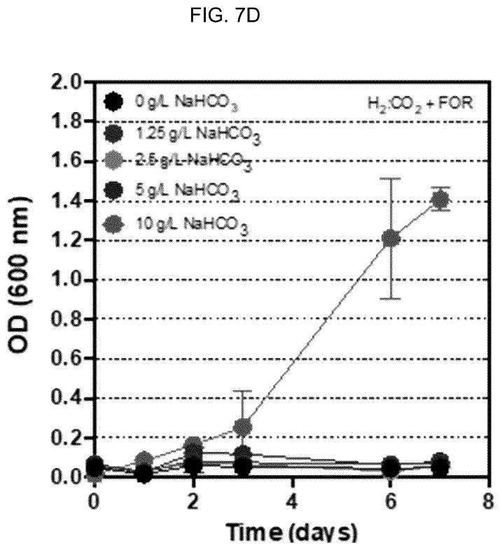

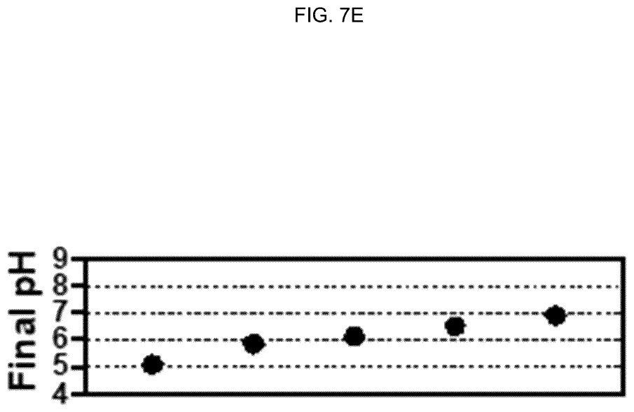

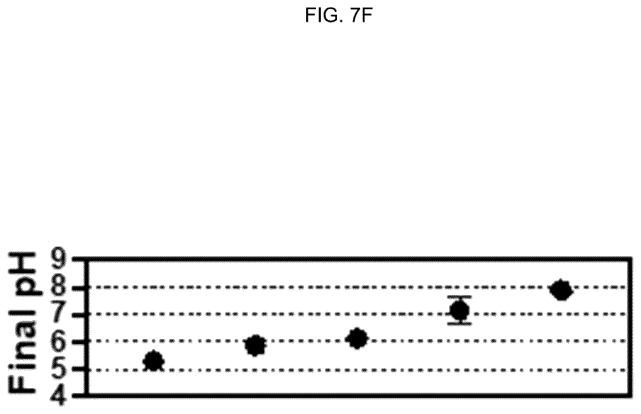

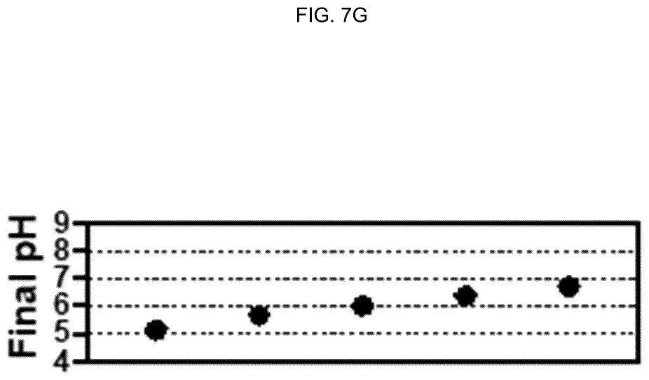

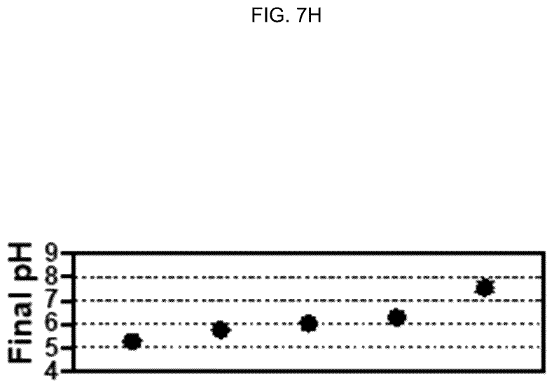

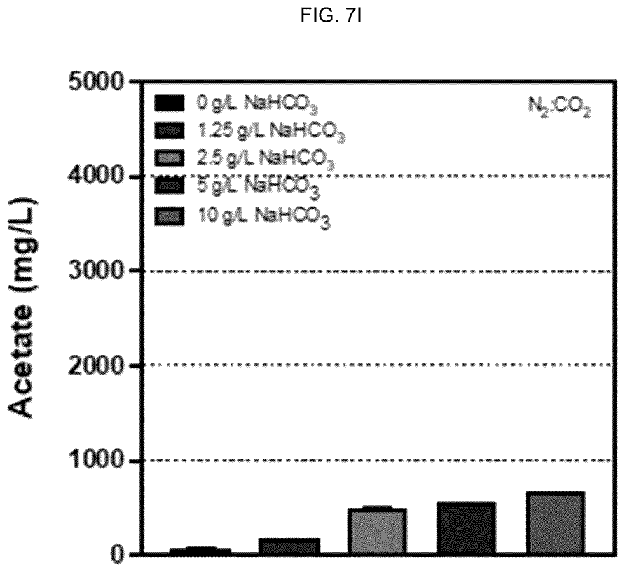

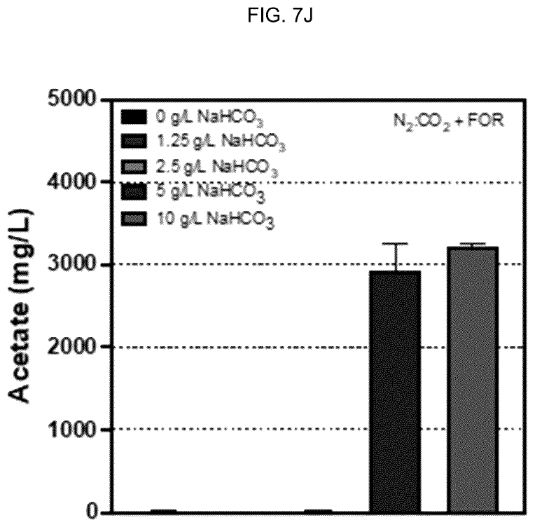

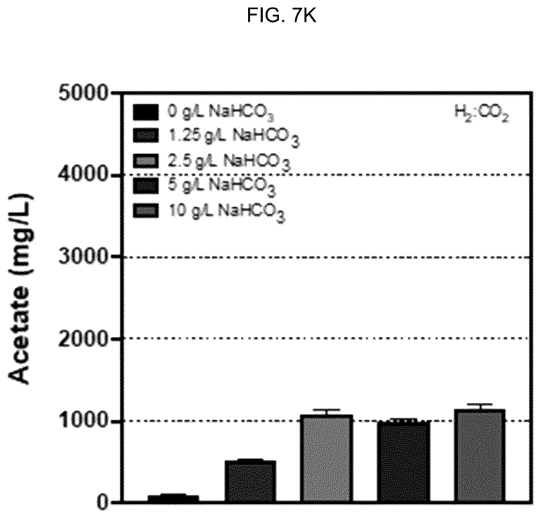

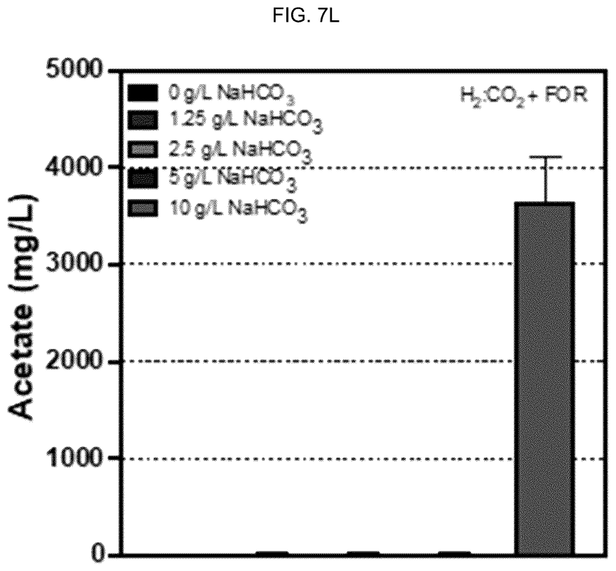

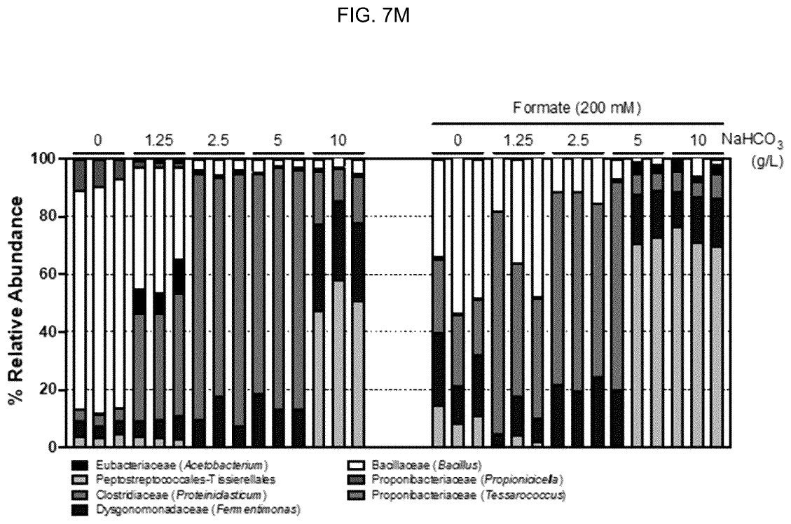

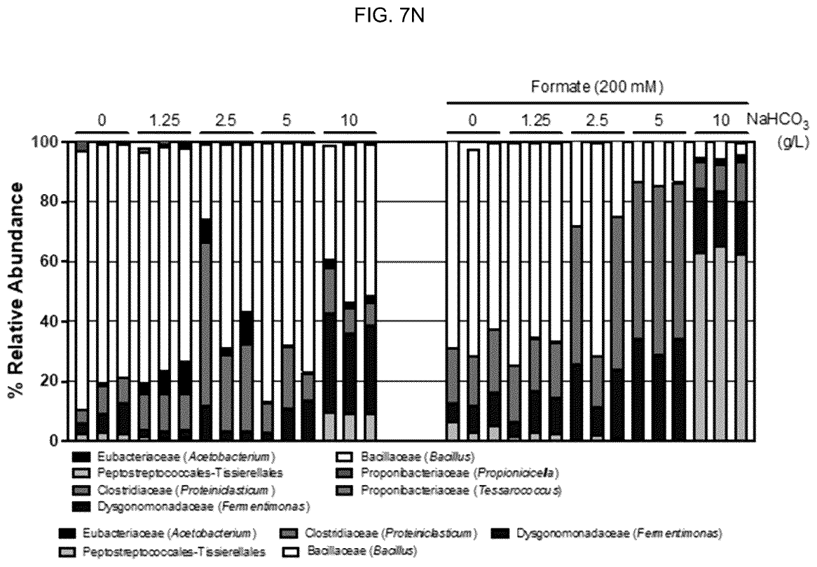

The original coal seam produced water microbiome consisted of a variety of taxa, including bacteria from families Eubacteriaceae, Smithellaceae, Fermentibacteraceae, Sulfurospirillaceae, Lentimicrobiaceae, Synergistaceae, Thioalkalispiraceae, Dysgonomonadaceae, and Dethiosulfatibacteraceae, and archaea from families Methanobacteraceae and Methanocorpusculaceae (FIGS. 7A-7M). Biocatalyst enrichment resulted in decreased diversity, which contained a minimal set of microorganisms comprised of Peptostreptococalles-Tissierellales, Clostridiaceae (Proteiniclasticum), Dysgonomonadaceae (Fermentimonas), Bacillaceae (Bacillus), and Eubacteriaceae (Acetobacterium).

In an embodiment, the invention provides a method for converting a carbon feedstock into more desirable products comprising: contacting a biocatalyst with the carbon feedstock, wherein the biocatalyst converts the carbon feedstock into the more desirable products, and wherein the biocatalyst comprises bacteria selected from the group consisting of Peptostreptococcales-Tissierellales, Proteiniclasticum, Proteiniphilum, Mesobacillus, Acetobacterium, and combinations thereof. In an embodiment, the biocatalyst comprises each of Peptostreptococcales-Tissierellales, Proteiniclasticum, Proteiniphilum, Mesobacillus, and Acetobacterium. In an embodiment, the method further comprises generating the biocatalyst, wherein generating the biocatalyst comprises: collecting a bacteria community in the wild; and manipulating the bacteria community collected from the wild to increase the concentration of bacteria comprising the biocatalyst. In an embodiment, manipulating the bacteria community collected from the wild comprises continual transfer on anaerobic media. In an embodiment, the carbon feedstock comprises at least one C1 compound. In an embodiment, the at least one C1 compound comprises CO₂, formate, methanol, CO, and combinations thereof. In an embodiment, the more desirable products comprise carbonaceous compounds selected from the group consisting of: butyrate, isovaleric acid, hexanoic acid, phenyl acetic acid, and combinations thereof. In an embodiment, the carbon feedstock is supplied as a gaseous stream comprising the C1 compound. In an embodiment, the carbon feedstock is supplied as a liquid. In an embodiment, the biocatalyst converts more than 80% of the C1 compound in the carbon feedstock into more desirable products. In an embodiment, the method is performed for a first batch wherein the carbon feedstock comprises a first C1 compound, followed by a second batch wherein the carbon feedstock comprises a second C1 compound that is different from the first C1 compound. In an embodiment, the biocatalyst comprises wild-type bacteria that are not genetically engineered. In an embodiment, the biocatalyst is configured to convert any of the C1 compounds into the more desirable products. In an embodiment, the biocatalyst is configured to adapt to different feedstocks without adjustment, wherein adapting to different feedstocks comprises an increase in concentration of bacteria best able to convert a particular feedstock relative to other bacteria comprising the biocatalyst. In an embodiment, the method generates between approximately 1 and approximately 7 g/L of acetate from the feedstock.

Having described the basic concept of the embodiments, it will be apparent to those skilled in the art that the foregoing detailed disclosure is intended to be presented by way of example. Accordingly, these terms should be interpreted as indicating that insubstantial or inconsequential modifications or alterations and various improvements of the subject matter described and claimed are considered to be within the scope of the spirited embodiments as recited in the appended claims. Additionally, the recited order of the elements or sequences, or the use of numbers, letters or other designations therefor, is not intended to limit the claimed processes to any order except as may be specified. All ranges disclosed herein also encompass any and all possible sub-ranges and combinations of sub-ranges thereof. Any listed range is easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as up to, at least, greater than, less than, and the like refer to ranges which are subsequently broken down into sub-ranges as discussed above. As utilized herein, the terms “about,” “substantially,” and other similar terms are intended to have a broad meaning in conjunction with the common and accepted usage by those having ordinary skill in the art to which the subject matter of this disclosure pertains. As utilized herein, the term “approximately equal to” shall carry the meaning of being within 15, 10, 5, 4, 3, 2, or 1 percent of the subject measurement, item, unit, or concentration, with preference given to the percent variance. It should be understood by those of skill in the art who review this disclosure that these terms are intended to allow a description of certain features described and claimed without restricting the scope of these features to the exact numerical ranges provided. Accordingly, the embodiments are limited only by the following claims and equivalents thereto. All publications and patent documents cited in this application are incorporated by reference in their entirety for all purposes to the same extent as if each individual publication or patent document were so individually denoted.

All numeric values are herein assumed to be modified by the terms “about” or “approximately,” whether or not explicitly indicated. The terms “about” or “approximately” generally refer to a range of numbers that one of skill in the art would consider equivalent to the recited value (e.g., having the same function or result). In many instances, the terms “about” and “about” include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

What is claimed is:

1. A method for converting a carbon feedstock into more desirable products comprising:
contacting a biocatalyst with the carbon feedstock, wherein the biocatalyst converts the carbon feedstock into the more desirable products, and wherein the biocatalyst comprises bacteria selected from the group consisting of Peptostreptococcales-Tissierellales, Proteiniclasticum, Proteiniphilum, Mesobacillus, Acetobacterium, and combinations thereof.

2. The method of claim 1 wherein the biocatalyst comprises each of Peptostreptococcales-Tissierellales, Proteiniclasticum, Proteiniphilum, Mesobacillus, and Acetobacterium.

3. The method of claim 1 wherein the method further comprises generating the biocatalyst, wherein generating the biocatalyst comprises:
collecting a bacteria community in the wild; and
manipulating the bacteria community collected from the wild to increase the concentration of bacteria comprising the biocatalyst.

4. The method of claim 3 wherein manipulating the bacteria community collected from the wild comprises continual transfer on anaerobic media.

5. The method of claim 1 wherein the carbon feedstock comprises at least one C1 compound.

6. The method of claim 5 wherein the at least one C1 compound comprises CO₂, formate, methanol, CO, and combinations thereof.

7. The method of claim 5 wherein the more desirable products comprise carbonaceous compounds selected from the group consisting of: butyrate, isovaleric acid, hexanoic acid, phenyl acetic acid, and combinations thereof.

8. The method of claim 5 wherein the carbon feedstock is supplied as a gaseous stream comprising the C1 compound.

9. The method of claim 5 wherein the carbon feedstock is supplied as a liquid.

10. The method of claim 5 wherein the biocatalyst converts more than 80% of the C1 compound in the carbon feedstock into more desirable products.

11. The method of claim 5 wherein the method is performed for a first batch wherein the carbon feedstock comprises a first C1 compound, followed by a second batch wherein the carbon feedstock comprises a second C1 compound that is different from the first C1 compound.

12. The method of claim 1 wherein the biocatalyst comprises wild-type bacteria that are not genetically engineered.

13. The method of claim 5 wherein the biocatalyst is configured to convert any of the C1 compounds into the more desirable products.

14. The method of claim 3 wherein the biocatalyst is configured to adapt to different feedstocks without adjustment, wherein adapting to different feedstocks comprises an increase in concentration of bacteria best able to convert a particular feedstock relative to other bacteria comprising the biocatalyst.

15. The method of claim 3 wherein the method generates between approximately 1 and approximately 7 g/L of acetate from the feedstock.
